(19) United States
(12) Patent Application Publication
(10) Pub. No.: US 2014/0224680 A1
(43) Pub. Date: Aug. 14, 2014

(54) KIT-OF-PARTS

(71) Applicants: Martin FOLGER, Ingelheim am Rhein (DE); Stefan LEHNER, Wiesbaden (DE); Thomas Adam DUCH, Gau-Algesheim (DE)

(72) Inventors: Martin FOLGER, Ingelheim am Rhein (DE); Stefan LEHNER, Wiesbaden (DE); Thomas Adam DUCH, Gau-Algesheim (DE)

(73) Assignee: BOEHRINGER INGELHEIM VETMEDICA GMBH, Ingelheim am Rhein (DE)

(21) Appl. No.: 14/176,316

(22) Filed: Feb. 10, 2014

(30) Foreign Application Priority Data
Feb. 11, 2013 (EP) 13154762.2

(51) Int. Cl. A61K 9/14; B65D 90/48

(52) U.S. Cl. CPC A61K 9/146; B65D 90/48
USPC 206/232

(57) ABSTRACT

The present invention relates to a kit-of-parts, comprising A) a packaging (5), containing a defined amount of a pharmaceutically active ingredient in the form of solid coated one or multi-layered particles; B) a container (10), comprising a liquid oily composition to disperse or suspend the solid coated one or multi-layered particles contained in A); C) a dispenser (20); D) an adapter (50), having an application arrangement piece (55) with a through-hole (57) to fit the dispenser (20) to the container (10) of B); and E) instructions for use of the kit. The user just needs to mix and shake the components in order to produce a stable homogeneous mixture, which is: suited for exact dosing; stable and reusable; convenient to use; safe for the user due to omittance of needles; and has no injury risk for the user.

### FIELD OF THE INVENTION

The invention relates to a kit-of-parts in the medical field, preferably veterinary medicine, particularly to a kit-of-parts intended for use in mammals, preferably companion animals, more preferred felines, in particular cats.

### BACKGROUND OF THE INVENTION

A variety of different therapies or combined therapies to treat the different diseases with different classes of compounds are known. Thus, the patient and particularly the owner or keeper of an animal is faced with a variety of different drugs and therapy forms available, all of which rely on different and in part demanding requirements. In case of an animal patient, it represents the burden of the owner or keeper of the animal to take care of the animal and to observe, control and provide the medication, usually on a daily basis. Therefore, there still exists a need for a system or combination of components which makes the treatment and prevention of diseases in mammals, preferably companion animals, more preferred felines, in particular cats, easier and more comfortable for the patient or owner or keeper of an animal.

Thus, the present invention aims at the improvement of the convenience of the treatment and/or prevention of diseases in mammals.

Furthermore, the selection of a suitable dosage form to be administered to a patient/an animal patient is of particular relevance. A tablet suffers from the deficiency that it cannot be divided in parts to allow an optimization of the dosage pattern individually. Therefore, a tablet is not the appropriate dosage form, especially when there is a necessity for precise dose adjustment. Therefore, it exists a need in prior art to provide a dosage form which allows for an accurate dosage.

As is known, the dose adjustment is easier by employing a liquid dosage form. Such a liquid dosage form is usually provided in sealed containers such as glass vials, often with a rubber stopper or seal which can be penetrated with a syringe assembly to obtain access to the content. The piercing of stoppers is typically achieved through the use of sharp, small-bored needles. However, a fundamental disadvantage of this conventional administration system for parenteral application is the necessity of using a syringe with a sharp needle. This exposes the user to the possibility of being accidently pricked by the syringe needle. In addition to the undesirable injury resulting from such an accidental needle prick, there may be a risk of contamination of the needle.

Furthermore, some liquid dosage forms suffer from the deficiency that the dosage form is more or less heterogeneous in the liquid carrier leading to a concentration gradient of the active ingredient(s) contained in the liquid carrier which is not desirable.

Besides, in the field of veterinary medicine there often occur problems in connection with the administration of a pharmaceutical active substance to an animal. Particularly, active ingredients accompanied with an unpleasant taste pose a severe problem because animals are mostly more sensitive to taste than humans. The use of flavors, e.g. meat flavor for predominantly carnivorous animals, in pharmaceutical compositions for animals is in many cases not sufficient to mask the unpleasant, e.g. bitter, taste of the active compound. Furthermore, animals, particularly cats, have the habit to break down their food by biting on it several times thereby destroying the dosage form such as tablets contained and release the unpleasant tasting drug. Thus, the present invention seeks to provide a dosage form wherein the taste of a pharmaceutically active compound may be masked in a suitable manner.

The following prior art documents could be identified:

US 2004/127846 relates to medical devices for mixing, preparing and administering therapeutic compositions, and more particularly to a system comprising two syringes and a locking ring wherein two compositions are mixed between the two syringes immediately prior to administration.
WO 98/36732 relates to a combination product for the oral administration of antibiotics as an aqueous suspension, comprising a) an active ingredient component in the form of particles coated with a polymeric, permeable coating agent capable of swelling and/or soluble in gastric juice; and b) a syrup base with a pH between 5 and 10 as a second component which is spatially separate from the first component a). The two components are presented in two separate containers packaged in a combined pack in the form of a “kit of parts”.
WO 2007/100779 relates to an adapter assembly for connecting a medication bottle to a needleless syringe, particularly a luer-lock syringe. The bottle adapter includes a cylindrically-shaped main body. Multiple annular fins project outwardly from the main body for engaging and gripping the interior surfaces of the bottle. This close interference fitting between the adapter and bottle provides a tight and effective seal. The adapter is particularly suitable for connecting a bottle containing a drug in solid form, such as a powdered drug, to a syringe containing a liquid carrier. The syringe is inserted into the adapter and liquid is injected into the bottle. The liquid mixes with the powdered drug to form a drug solution. The bottle adapter can be used in a wide variety of medical, dental, pharmaceutical, and other healthcare applications.
US 2004/014795 relates to the new use of bradycardiac substances such as a Ca^{2°} channel blocker, beta-receptor blocker or I_{f} channel blocker, the I_{f} channel blockers being preferred, optionally in combination with a cardioactive substance for inducing the regression of myocardial diseases accompanied by hypertrophy, particularly for the treatment of idiopathic hypertrophic cardiomyopathies (HCM) in humans and domestic pets.
WO 2011/098582 relates to novel crystalline forms of ivabradine hydrochloride and pharmaceutical compositions prepared therefrom.

Therefore, the problem underlying the present invention is to provide a dosage form which allows for an accurate dosage and avoids a concentration gradient of the active ingredient(s), whereby the dosage form is sufficiently stable over time, the conditions for the treatment and/or prevention of mammal patients is simplified, and the comfort of the treatment is improved. Optionally, it shall be possible to provide the dosage form in a taste masked form, wherein the taste of a pharmaceutically active compound may be masked in a suitable manner which allows to avoid administration problems in the treatment and/or prevention of diseases, for example heart disease, in mammal patients, preferably companion animals, more preferred felines, in particular cats.

### DESCRIPTION OF THE INVENTION

The above-mentioned problems are surprisingly solved according to the subject matter of the independent claims of the present invention. Preferred embodiments are the subject of the sub-claims.

Therefore, according to the present invention it is provided a kit-of-parts comprising or consisting of
A) a packaging, containing (a defined amount of) a pharmaceutically active ingredient in the form of solid coated one or multi-layered particles;
B) a container, comprising a liquid oily composition (to disperse or suspend the solid coated one or multi-layered particles contained in A), preferably in a homogenous mixture);
C) a dispenser;
D) an adapter, having an application arrangement piece with a through-hole to fit the dispenser to the container of B); and
E) instructions for use of the kit.

Furthermore, the following variations are preferred embodiments of the present invention:
i) The container as described under B) supra can optionally be placed into a bag of a foil packaging, preferably aluminum foil, and the opening can optionally be completely sealed so that the container according to B) supra is completely surrounded by a tight foil of foil package, preferably aluminum. This packaging process is known as pouching and the preferred type of additional packaging is called an aluminum pouch.
ii) As an optional alternative to the container described under B), the liquid oily composition can be directly filled into a flexible container made of foil packaging, preferably aluminum foil. The liquid oily composition can optionally be filled into an aluminum bag, and the bag can optionally be tightly sealed after filling. The process is also known as pouching and the preferred primary packaging container is called an aluminum pouch.
iii) The closure which is used in conjunction with the container as described under B) supra can optionally contain an additional insert or disk which will further improve the tight closing of the container-closure system used.
iv) The container which is described under B) supra can optionally consist of a bottle with an opening mouth (see (11) in FIG. 2). In addition to the configuration as described in FIG. 2, a sealing layer can optionally be added after filling which is placed on the mouth of the bottle during storage. This seal needs to be removed before adding the adapter [see (50)].
v) The container which is described under B) supra consists preferably of glass. Besides the suitability for pharmaceutical use, different types of glass can be used for this container, such as glass with a surface treatment as well as glass with protective layers. These are examples for preferred glass materials with low ion leaching properties, other examples are however possible.

The invention is also related to a kit-of-parts for the use in a method of treating and/or preventing a heart disease in a mammalian patient, preferably in a companion animal, more preferred in a feline, in particular in a cat.

Furthermore, the invention is directed to a method for administering a pharmaceutically active ingredient in the form of solid coated one or multi-layered particles, comprising or consisting of the steps of:
a) opening of a container (10) comprising a liquid oily composition;
b) opening of a packaging (5) comprising (a defined amount of) the active ingredient in the form of solid coated one or multi-layered particles;
c) adding the whole content of the packaging into the container;
d) applying an adapter, having an application arrangement piece with a through-hole onto the container;
e) closing the container;
f) shaking the container;
g) opening the container;
h) inserting the tip of a dispenser into the through-hole of the application arrangement piece of the adapter;
i) removing liquid from the container, using the dispenser;
j) disconnecting the dispenser from the adapter;
k) administering the liquid with/by means of the dispenser to the patient/animal or to patient/animal food;
l) closing the container;
m) optionally repeating steps f) to l);
   whereby the sequence of step a) and step b) may also be reversed,
   whereby the sequence of step k) and step l) may also be reversed.

In case the preferred configuration with an aluminum pouch is applied as described under i) supra, the pouch needs to be opened first before starting with step a) above. All other following steps remain unchanged.

In case the preferred configuration with an additional seal is applied as described under iv) supra, the seal needs to be opened after step g) above before continuing with step h) above. All other previous and following steps remain unchanged.

The kit-of-parts according to the present invention allows the user to readily produce the liquid dosage form to be administered to the mammal by opening the container comprising a liquid oily composition, opening of the packaging containing a defined amount of a pharmaceutically active ingredient in form of solid coated one or multi-layered particles, adding the solid coated one or multi-layered particles, which are optionally or preferably taste masked, of the packaging into the container, applying an adapter onto the container and closing the container. After shaking the container in order to provide a homogenous mixture, and again opening the container, a dispenser, preferably a syringe-like dispenser, adapted to fit into the adapter on the container is used to remove the homogeneous mixture from the container. After disconnecting the dispenser, preferably the syringe-like dispenser, from the adapter the homogeneous mixture may be administered to the patient/animal or to patient/animal food. Then the container is closed again. It is a matter of course that the first two steps (a, b) and last two steps (k, l) may be performed in reversed order so that the container is closed and subsequently the liquid formulation is administered to the patient/animal or food.

Furthermore, the procedure starting with the shaking of the container to the administration of the liquid formulation and closing of the container (f to l) may be optionally repeated several times.

Surprisingly, the user just needs to mix and shake the components in order to produce a stable homogeneous mixture, which:
is suited for exact dosing of solid coated one or multi-layered particles in a liquid,
is stable and reusable,
is convenient to use,
is safe for the user due to omittance of needles,
has no injury risk for the user.

Thus, the present invention provides an advantageously improved system that permits the withdrawal/removal of a liquid dosage form from a container without requiring the use of a syringe having an exposed, sharp needle. It provides simple and rapid access to the liquid medicament included within the container. Additionally, the present invention constitutes an improved system, wherein the components are readily available either commercially available or may be manufactured at very low cost, e.g. with mass production techniques.

Furthermore, solid coated one or multi-layered particles (e.g. pellets or cellets) can be advantageously used to taste-mask the active ingredient, to increase the stability of the active ingredient or to modify the release (e.g. extended or sustained release) of the active ingredient. However, the exact dosing and administration of such liquids containing solid coated one or multi-layered particles (e.g. pellets or cellets) is technically challenging due to the tendency of the solid coated one or multi-layered particles (e.g. pellets or cellets) to sedimentation. The present invention surprisingly offers a solution to this challenge by providing a stable homogenous mixture, which can be even reused several times within a time frame of 4-8 weeks after initial mixture.

### DETAILED DESCRIPTION OF THE INVENTION

In the following components A) to E) of the kit-of-parts according to the present invention will be described in detail.

### Component A)

According to the present invention component A) of the kit-of-parts represents a packaging containing a defined amount of a pharmaceutically active ingredient in the form of solid coated one or multi-layered particles.

The packaging is not limited according to the present invention. Any pharmaceutically acceptable and suitable packaging may be used to incorporate a solid formulation. The form and size of the packaging may be arbitrarily selected in order to include the defined amount of the pharmaceutically active ingredient in a solid form to be present in the kit-of-parts. Exemplarily mentioned are foil packages, particularly made of thin flexible foil(s), for example, laminated foil(s), bags, pouches, sacs, tubes and the like. The materials of the package are chosen in line with the pharmaceutical requirements and restrictions well known to the person skilled in the art. A particularly preferred material is selected from aluminum foil, plastic materials, paper and the like. The packaging material may also represent a combination of several materials, for example an outer wrapper comprising one type of material and an inner wrapper comprising another type of material.

The material of the packaging is preferably made up of a moisture-proof material which protects the included formulation and prevents the ingress of humidity. Preferably the packaging is due to a better handling performance selected from one or more flexible materials. Also preferred is the use of child-proof materials.

The contained pharmaceutically active ingredient in the form of solid coated one-layered or multi-layered particles is preferably selected from the class of I_{F}-channel blockers. “I_{F}-channel blockers” also referred to as “funny current inhibitors” or “I_{F}-inhibitors” are those chemical compounds that interact with and inhibit the I_{F}-channel. They are believed to be useful for treating and even inducing the regression of myocardial diseases associated with hypertrophy, in particular for treating idiopathic hypertrophic cardiomyopathies (HCM) in domestic animals.

Useful compounds are disclosed by EP 0 065 229 B 1, especially zatebradine, and U.S. Pat. No. 3,708,485, especially alinidine.

Very useful and preferred compounds are disclosed in EP 0 224 794 B1, among which the preferred I_{F}-channel blocker is (+)-3-[(N-(2-(3,4-dimethoxy-phenyl)ethyl)-piperidine-3-(S)-yl)-methyl]-(7,8-dimethoxy-1,3,4,5-tetrahydro-2H-3-benzazepin-2-one which is also useful for the treatment and/or prevention of heart failure. It has been given the international nonproprietary name (INN) cilobradine. The hydrochloride form is called cilobradine hydrochloride.

Cilobradine (3-[(N-(2-(3,4-dimethoxy-phenyl)-ethyl)-piperidine-3-yl)-methyl]-(7,8-dimethoxy-1,3,4,5-tetrahydro-2H-3-benzazepin-2-one) and its hydrochloride salt, are disclosed for example in EP 0 224 794 B1 and its US counterpart U.S. Pat. No. 5,175,157. Cilobradine is also known to have a favorable activity in the treatment or prevention of heart failure (see EP 1 534 296 B1). Cilobradine, zatebradine and alinidine are also known to have a favorable activity in the treatment and induction of the regression of idiopathic hypertrophic cardiomyopathy (HCM), ischemic cardiomyopathy and valvular hypertrophic heart diseases (see WO 01/78699).

Alinidine [2-(N-allyl-2,6-dichloro-anilino)-2-imidazolidine] is disclosed, for example, in U.S. Pat. No. 3,708,485, and ivabradine 3-[3-[[[(7S)-3,4-dimethoxybicyclo[4.2.0]octa-1,3,5-trien-7-yl]methyl]methylamino]propyl]-1,3,4,5-tetrahydro-7,8-dimethoxy-2H-3-benzazepin-2-one and its hydrochloride salt is disclosed, for example, in EP 0 534 859 B1.

Zatebradine [1-(7,8-dimethoxy-1,3,4,5-tetrahydro-2H-3-benzazepin-2-on-3-yl)-3-[N-methyl-N-(2-(3,4-dimethoxy-phenyl)-ethyl)-propane] is disclosed, for example, in EP 0 065 229 B1 and its US counterpart U.S. Pat. No. 5,516,773. Zatebradine is known to have a favorable activity in the treatment of cardiac insufficiency (see EP 0 471 388 B1).

Ivabradine is especially known to have a favorable activity in the treatment of myocardial disorders (from EP 0 534 859 B1 or U.S. Pat. No. 5,296,482). The administration of ivabradine in cats with subclinical HCM is described by Riesen et al. (Riesen, Schober, Smith, Otoni, Xiaobai, Bonagura; AJVR, Vol. 73, No. 2: Effects of ivabradine on heart rate and left ventricular function on healthy cats and cats with hypertrophic cardiomyopathy).

The administration of an I_{F}-channel blocker, as a component of the kit-of-parts, to a mammal patient, in particular to cats, preferably results in not just the treatment and/or prevention of heart disease, particularly heart failure, but also to an improved quality of life and/or general health condition and/or to a prolonged life expectancy. With regard to the meaning of “improved quality of life”, “improving general health condition”, “prolonging life expectancy” “long-term survival time” or “reducing cardiac mortality or morbidity”, it is referred to WO 2013/024036 (PCT/EP2012/065704), the whole disclosure thereof is incorporated by reference in the present disclosure.

Therefore, the use of an I_{F}-channel blocker as pharmaceutically active ingredient of the solid coated one or multi-layered particles contained in the packaging as part of component A) of the kit-of-parts has in all probability an overall positive effect on the whole body of the animal patient, including health and demeanor such as the activity level and the mind of the patient.

Thus, the pharmaceutically active ingredient is preferably selected from cilobradine, alinidine, zatebradine and ivabradine, preferably zatebradine or its enantiomer cilobradine, even more preferred cilobradine and cilobradine hydrochloride, present in the solid coated one or multi-layered particles.

However, also other pharmaceutically active ingredients than an I_{F}-channel blocker may be used.

According to the present invention the solid coated one or multi-layered particles are not limited. Any particles coated with one, two, three, four or more layers may be used. The expression “particles”, according to the present invention, are overall solid pieces. They provide the pharmaceutically active ingredient in a suitable solid formulation in package A). In the finally provided liquid dosage form (in container B)) the particles make up the solid phase of a dispersion or suspension in an overall pharmaceutical liquid oily composition. For the sake of better handling the particles are preferably free-flowing.

The particles of the solid formulation are preferably sized in a way to generate an acceptable mouth feel for the patient, e.g. the cat, meaning: they are not too big as that would lead to a gritty sensation which would lower the acceptance by the patient/animal. This may be also of importance with regard to the present liquid dosage form which is orally administered or added to the food of the patient/animal.

According to the present invention the particles are solid coated one or multi-layered particles. That is the particles comprise or consist of an inert core and one or more layers. Within the context of the present invention the multi-layered particles are particles coated with more than one layer.

The solid coated one or multi-layered particles are understood to be synonymous to pellets. The term “pellets” is to be understood in the present invention in the sense that a drug or drug-loaded particles is/are coated with one or multiple layers of polymers. The coating(s) of the particles may have an additional function such as taste masking of a bitter tasting pharmaceutically active ingredient. The coating(s) can also be used to enhance the stability of the finally formulated one or multi-layered particles or the coating(s) may provide modified release. Other possible functional coatings which may be used are those already known by the person skilled in the art.

More preferably the solid coated one or multi-layered particles are cellets. Cellets comprise or consist of cellulose such as microcrystalline cellulose in the inert core which is coated with one or more layers. Particularly cellets have advantages with respect to the processing and the physical properties of the finally formulated one or multi-layered particles.

According to one embodiment the solid coated one or multi-layered particles are taste masked. “Taste masked”, in the sense of the invention, means that the molecules with an unpleasant taste are hidden away from the patient's perception, covered with another, more pleasant taste or flavor or according to the well-designed architecture in the one or multi-layered particles. It is to be understood as a functional term, especially with respect to the natural taste perception of an animal.

In case the pharmaceutically active ingredient is selected to be an I_{F}-channel inhibitor, in a preferred embodiment of the present invention the solid coated one or multi-layered particles contained in package A) comprises 0.001 to 0.2 mg of the I_{F}-channel inhibitor or the pharmaceutically acceptable salt thereof per mg of the solid coated one or multi-layered particles. The solid coated one or multi-layered particles containing the pharmaceutically active ingredient in form of the I_{F}-channel inhibitor are preferably present in package A) in such an amount so that a final concentration of 0.1 to 20 mg/ml, preferred 0.5 to 5 mg/ml, more preferred 0.5 to 4 mg/ml, most preferred 0.5 to 3 mg/ml of the pharmaceutically active ingredient in form of the I_{F}-channel inhibitor will be obtained in the oily composition in container B) as well as in the preferably syringe-like dispenser C).

According to a mode of the present invention, it is particularly preferred to use multi-layered particles in the packaging of component A) of the kit-of-parts. A particularly preferred embodiment thereof will be described later in the present description in detail.

### Component B)

As component B) of the kit-of-parts according to the present invention a container is provided, comprising a liquid oily composition to disperse or suspend the solid coated one or multi-layered particles contained in the package A) described-above. The container is closed with a sealing over the opening thereof. The container is sealed in a usual manner so as to prevent the outflow of the content. The type of sealing of the container is not limited according to the present invention, any type of sealing known for the skilled person and used in the pharmaceutical sector may be used. Preferably the container comprises a type of seal such as the non-limiting examples of a cap, closure, plug, stopper, more preferably a screw cap is used. In addition, the open mouth of the bottle can be optionally sealed with a seal layer consisting of materials known to the skilled person which is removed before the first use. For example, the container includes a threaded female connection at the upper end that cooperates with a male connection of a screw-top cap to securely connect both. Other types of sealing mechanism are also possible. In addition, a child-proof lock may be used.

According to a preferred embodiment of the present invention the sealing provided on the container is designed and dimensioned in such a manner that the container may be sealed again with the sealing if an adapter (explained below) is applied on the opened container, whereby the sealing is in a usual manner so as to prevent the outflow of the content. It is preferred that the sealing of the container comprises an additional gasket seal which is adapted to seal the container in liquid-tight manner also in case the adapter is already applied onto the container.

According to another embodiment of the present invention the liquid content can optionally be filled into a flexible pouch, preferably an aluminum pouch.

Within the context of the present invention the container is not particularly limited. Any suitable container may be used. The container comprises a hollow body preferably having a flat basement in order to place the container in a stable manner on a surface such as a table. Exemplarily mentioned are bottles, flasks, vials, ampoules, pharmaceutical phials and the like, particularly preferred is a glass vial or glass bottle. According to the present invention a vial is rather to be understood in the sense of a little bottle or a flacon then an ampoule. The material of the container may be selected among materials which are known to be suitable for the packaging in the pharmaceutical sector. Particularly preferred are plastic materials and glass, most preferred is glass due to the transparency and compatibility with a variety of pharmaceutical formulations. The material glass has the advantages that it is inert and has practically no negative impact on the content of the container. Special types of glass surface-coated glass with reduced potential for leaking ions can optionally be used when required.

In case pouching is chosen as the primary packaging concept, aluminum is the preferred material for the pouch.

According to a preferred embodiment of the present invention the container B) is a glass vial or a glass bottle. The vial or bottle can be a pharmaceutical vial or bottle of known construction. Especially this package form has turned out to be useful for the storage of the overall liquid, oily pharmaceutical composition because it strongly inhibits the diffusion of humidity through the walls of the respective vial or bottle into the overall water-free pharmaceutical composition.

It is further preferred that the glass vial or bottle comprises or consists of a dark glass, e.g. green or even better brown glass is used, in order to further protect the ingredients, e.g. against light.

The dimensions and shape of the container may be selected according to containers known and used in the pharmaceutical field as well as the type of patient/animal to be treated and the intended dose regimen. Appropriate sizes for such glass vials or bottles can be developed by a person skilled in the art with respect to the amount to be filled in, e.g. for single use, for providing an appropriate amount for the complete therapy of one patient or for the treatment of a patient group, for example a breed of domestic animals.

The container B) is filled with an overall liquid, oily composition. The composition comprises any oily carrier(s) which may be used in a pharmaceutical composition that is an oily or oil compound or a mixture of several oily or oil compounds which are suitable for consumption by a patient/an animal patient. Various types of oily or oil compounds or mixtures thereof may be used. The preferred liquid oily composition in the container comprises or consists of

a) an edible oil, such as a vegetable oil, preferably selected from linseed oil, peanut oil, sunflower oil, olive oil, safflower oil or the like; or
b) an animal oil such as fish oil, or
c) one triglyceride or a mixture of several triglycerides, or
d) mixtures thereof,
preferably the container comprises or consists of a mixture of triglycerides, more preferably of medium chain triglycerides.

For instance edible oils may be employed. Examples of usable oil compounds are vegetable oils, for example linseed oil, peanut oil, sunflower oil, olive oil, safflower oil and the like; animal oils such as fish oil and the like. Instead of olive oil which is known to have a strong inherent smell, compounds which are less sensitive to oxidation may be used such as triglycerides, preferably medium chain triglycerides and mixtures thereof, such as MIGLYOL®. Therefore, it is advantageous and accordingly preferred to use triglycerides as basis.

The oily composition is a carrier or a mixture of carriers in order to disperse or suspend the solid coated one or multi-layered particles or pellets containing the pharmaceutically active ingredient, such as the IF-channel inhibitor. In the framework of the present invention a “dispersion” is to be understood in its broadest sense to mean a system in which particles are dispersed in a continuous phase of a carrier or mixture of carriers. A dispersion is classified in a number of different ways, including how large the particles are in relation to the particles of the continuous phase. A dispersion usually comprises a coarse dispersion such as a suspension, a colloid dispersion as well as a solution. However, according to the present invention a solution is not preferred and not intended to be achieved in order not to damage the solid coated one or multi-layered particles or pellets used. Therefore, the carrier composition is selected to be an oily composition, wherein the essential part thereof is derived from one or more oils or oily compounds. As a result, the pharmaceutical active ingredient is preferably selected from ingredients which are not soluble in an oily composition such as the class of IF-channel inhibitors which is soluble in water but not soluble in an oily composition, so that a dispersed system in form of a suspension or colloid dispersion is intended and not a true solution. Therefore, if the solid coated one or multi-layered particles or pellets are added to the oily composition, a dispersion, preferably a suspension is obtained.

Therefore, the administration mode used in the present invention is an overall liquid dosage form. A liquid dosage form is particularly advantageous because this ensures a very precise dosing of the respective drug.

Since the solid coated one or multi-layered particles or pellets are to be incorporated into a liquid matrix, i.e. the overall liquid oily composition it has to be ensured that the liquid oily composition does not interact with the particles/pellets in a way that would compromise the desired drug release profile. The skilled person knows how this may be achieved. For example, another protective layer onto the surface of the particles may be added. Another approach is the addition of one or more further excipients into the liquid oily composition, for example in order to increase the viscosity of the oily composition, as will be explained below. It may be preferred to combine the respective teachings.

According to a preferred embodiment one or more excipients are preferably added into the liquid oily composition in order to increase the viscosity of the oily composition. As a result, stabilizing of the obtained suspension by increasing the viscosity is achieved and at the same time sedimentation and caking of the particles is avoided.

Therefore, it is preferred according to the present invention that the oily composition comprises one or more viscosity enhancers selected from the following: silicon dioxide(s), preferably colloidal silicon dioxide(s), e.g., hydrophilic colloidal silicon dioxide, hydrophobic silicon dioxide, EC (cellulose ether), poly(1-vinylpyrrolidin-2-one) (PVP), aluminum stearate, xanthan gum, carrageen, and/or starch derivatives.

Further, it is preferred that the oily composition contains colloidal silicon dioxide(s), e.g. hydrophilic colloidal silicon dioxide and/or hydrophobic colloidal silicon dioxide in order to optimize the physicochemical properties of the composition. Commercially available silicon dioxides (silica) are for example AEROSIL® 200 (hydrophilic) and AEROSIL® R972 (hydrophobic), both sold under these respective trade names by Evonik Röhm, Darmstadt, Germany.

Especially preferred is that the liquid oily composition in container B) comprises a colloidal silicon dioxide, e.g. hydrophilic colloidal silicon dioxide, and optionally hydrophobic silicon dioxide, more preferably a mixture of colloidal silicon dioxide, e.g. hydrophilic colloidal silicon dioxide, and hydrophobic silicon dioxide which ensures an appropriate viscosity behavior of the oily composition. This appropriate viscosity behavior is also maintained if the solid formulation is present in the oily composition. That is, the oily composition as well as the dispersion or suspension obtained after the addition of the solid coated one or multi-layered particles or pellets into the oily composition, remains more or less unchanged over the storage period. Surprising the stability of the obtained suspension or dispersion is maintained over a period of about 4 to 8 weeks.

Therefore, during storage, the oily composition and likewise the obtained dispersion, particularly suspension, preferably exhibits a high viscosity. Such a high viscosity also prevents the sedimentation of the suspended particles in the oily composition. If shaken, however, the viscosity of the suspension might transiently be lowered so that it can easily be applied via an oral dispenser. This behavior (thixotropy) can for example be reached by the mentioned mixture of colloidal silicon dioxide, e.g., hydrophilic colloidal silicon dioxide, and hydrophobic colloidal silica.

More preferably the liquid oily composition comprises or consists of a mixture of medium chain triglycerides, colloidal silicon dioxide such as a hydrophilic colloidal silicon dioxide as viscosity enhancer, optionally a hydrophobic colloidal silicon dioxide and preferably one or more flavoring agents such as meat flavor.

Especially preferred are oily pharmaceutical compositions that comprise a mixture of colloidal silicon dioxide, e.g. hydrophilic colloidal silicon dioxide and hydrophobic colloidal silicon dioxide, preferably at a weight percent ratio of 0.5:1 to 50:1, more preferred 1:1 to 25:1, more preferred 2:1 to 10:1, most preferred 2.25:1 to 5:1.

A person skilled in the art is able to vary the respective silicon dioxide concentrations within these ranges. Regularly possible and preferred is such a ratio and content of both compounds that gives the suspension a very advantageous physicochemical behavior, i.e. a high viscosity during storage and a lowered viscosity after shaking due to the mechanical energy exerted by the shaking. This effect stabilizes the suspension during its storage but allows that it can easily be applied after shaking, e.g. by a dispenser, preferably a syringe-like dispenser in order to administer it directly to a patient/an animal or by dropwise addition to a food preparation etc.

Furthermore, the obtained dispersion, particularly suspension, being stable over an extended period of time, provides a homogeneous mixture of the solid coated one or multi-layered particles or pellets in the liquid oily composition. In the context of the present invention the mixture is considered to be homogeneous if the achieved mixture is uniform in composition, i.e. the solid coated one or multi-layered particles are distributed uniformly in the liquid matrix. As a result, a concentration gradient of the pharmaceutically active ingredient in the oily matrix is avoided. Disadvantageous effects due to different concentrations present in the liquid formulation do not occur.

It is further preferred that the oily composition contains a flavoring agent in order to render it attractive for the patient, e.g. a meat flavor to render it attractive to carnivorous animals like cats.

If using cilobradine hydrochloride as pharmaceutically active ingredient it is particularly preferred to provide the solid coated one or multi-layered particles or pellets in such an amount so that these are suspended in the liquid oily composition to an extent of about 3.0 to 4.5%, preferably about 3.2 to 4.0%, particularly preferred about 3.8% (w/v), resulting in a concentration of 2 mg/ml of the pharmaceutically active ingredient.

### Component C)

As component C) of the kit-of-parts according to the present invention a dispenser, preferably syringe-like dispenser is provided. Such a preferred syringe-like dispenser or oral dispenser preferably includes a syringe barrel having an elongated cylindrical body defining a chamber for retaining fluid, a plunger in fluid-tight slidable engagement inside the barrel, an elongated plunger rod extending in the longitudinal direction through the barrel, and a tip having a tip passageway therethrough in fluid communication with the chamber. Furthermore, the preferred syringe-like dispenser comprises a usually known finger support or flange. In the dispenser, preferably syringe-like dispenser, a needle is not attached to the tip of the dispenser. Therefore, the dispenser allows a needleless access to a container with a blunt penetrating tip.

The function of the dispenser, preferably syringe-like dispenser, is like said of a common syringe so that the desired amount of liquid is withdrawn into the chamber by moving the plunger rod in a longitudinal direction with respect to the barrel.

In the first line any form and size may be used which is derivable from a usual syringe wherein the needle is omitted. The material of the dispenser is selected from a plastic material usually employed in the pharmaceutical field. The dispenser may be a usual dispensing pipette, which has a plunger and a plunger rod, as available from Hubert de Backer (St. Niklaas, Belgium).

According to a preferred embodiment the dispenser of the kit-of-parts is designed and dimensioned in such a way that the contained fluid allows a simplified dose adjustment for a patient/an animal patient to be administered.

In order to achieve such a simplified dose adjustment the front surface of the dispenser is preferably provided with a scale reading which facilitates to determine and adjust the amount of pharmaceutically active ingredient to be administered to the patient.

Preferably the labeling indicated on the dispenser contains marks which correspond to the weight (in kg) of the patient/animal patient. For example, the dispenser is subdivided with a division scale composed of different marks. The marks stand for the kilograms of the patient/animal patient. For example, the division scale may be classified in 10 different marks from 1 to 10 kg, each mark represents the amount in kg, respectively increasing 1 kg from mark to mark. Therefore, in case the user has a cat having 4 kg weight, the dispenser shall be filled with the pharmaceutical liquid dosage form up to the mark ‘4 kg’ in order to provide the correct dose to be administered.

Therefore, the pharmaceutical liquid dosage form is, for example, filled into the dispenser according to animal's weight and provides the correct dose to be administered to the animal. The marks depend from the pharmaceutically active ingredient to be used, the concentration and dose of the pharmaceutical liquid dosage form to be adjusted as well as the patient/animal and the disease to be treated. The suitable dose of a pharmaceutically active ingredient is per se known by those skilled in the art. Taking the example of the I_{F}-channel inhibitor as pharmaceutically active ingredient into account, preferably cilobradine, or a pharmaceutically acceptable salt thereof, the dose is adjusted preferably based on the rule that an I_{F}-channel inhibitor or a pharmaceutically acceptable salt thereof is to be administered in a daily dosage of 0.01 to 2 mg/kg bodyweight.

It is also possible to indicate “kg” on the dispenser to inform the user about the respective function. For the sake of information and/or design and in case an animal should be treated an illustration of the animal to be treated, for example a cat, may be applied on the surface of the dispenser, too.

According to a further preferred embodiment the plunger of a dispenser, preferably syringe-like dispenser has a protruding part which extends in the direction of the tip and fits within the tip passageway in such a manner to be able to press the liquid in the tip passageway practically completely out of the dispenser. This embodiment ensures that the liquid dosage form filled in and contained in the dispenser is in total administered to the animal to be treated. Additionally no product is remaining in the dispenser after administration which could be contaminated later on.

Therefore, the dispenser allows to improve and to simplify the administration for the user and also facilitates the determination of the dose to be given to the patient/animal patient. The steps of finding out and determining the exact dose for each patient/animal to be treated may be omitted. The oral dispenser makes it also possible that different animals, for example a number of cats, may be easily treated without the observation of a complicated treatment schedule. It is therefore more comfortable for the owner or keeper of an animal or more than one animal to administer the dosage form to the animal(s), particularly with regard to several administrations per day to be repeated over a long period of time.

It is preferred that the dispenser is provided with a sealing such as a cap after use in order to avoid contamination of the interior of the dispenser, particularly in case of a repeated use thereof. A sealing of the dispenser is also advantageous in order to prevent the ingress of humidity, particularly in case the pharmaceutically active ingredient is sensitive against moisture.

### Component D)

Component D) of the kit-of-parts according to the present invention is an adapter having an application arrangement piece with a through-hole to fit the dispenser C) to the container B). The application arrangement piece with a through-hole provides access to the content of the container.

The form of the adapter is adapted to the form of the opening and mouthpiece of the container so that the adapter is a removable seal disposed on, and sealingly engaged with the opening and mouthpiece of the container B). Therefore, the adapter is formed to couple the container with the dispenser, preferably syringe-like dispenser. Preferably the adapter comprises a type of sealing such as a cap, closure, plug, stopper, preferably a stopper. Other types of sealing mechanisms are also possible.

The adapter may be a usual adapter of a dosing pipette as available from Stella (Eltville, Germany).

The adapter, preferably a stopper, may be positioned into the mouthpiece of the container and is formed in such a way that a part of the outer circumference of the adapter and a part of the inner periphery of the mouthpiece of the container is provided in closely fitting manner. Preferably the adapter engages with the container in order to seal with the inner walls of the container in a fluid-tight manner. The adapter further includes an application arrangement piece with a through-hole which extends from a surface, preferably an end surface, of the adapter and, in mounting position, extends in the opposite direction to the basement of the container. In the connecting region between the adapter and the container, the form and dimension of the adapter are adapted to the opening and mouthpiece of the container.

Preferably the outer contour of the adapter as seen in the circumferential direction is selected in such a manner that the adapter has an oversize and may be pressed into the opening of the container, in an alternative manner a form-fitting connection may be provided. For example, the adapter may comprise at least in a sub-section, one or more, continuously or intermittently formed, protrusions in circumferential direction extending all around or extending in part thereof.

Furthermore, the adapter preferably has an additional edge, preferably an edge protruding to the outside as a further contact surface to provide additional contact with the container, particularly with the upper side or rim of the mouthpiece, in order to provide a tighter fit and to improve the fitting accuracy with the container.

The application arrangement piece with a through-hole may protrude from the remaining part of the adapter but this is not necessary in any case. Preferably the adapter is provided as a rotationally symmetrical element having a circular cross-section. Other arrangements are also possible.

In order to remove the liquid dosage form from the container the dispenser is inserted into the application arrangement piece having a through-hole. The application arrangement piece, particularly the through-hole, is preferably formed to accommodate and surround the tip of the dispenser. The form and size of the through-hole as well as the form and size of the tip are particularly matched to each other in such a way that the withdrawing of the liquid dosage form is possible without leakage of the content of the container B), whereby the container is held upside down with the opening downwards. That is, the exterior surface of the tip of the dispenser is cylindrical and the interior surface of the through-hole is cylindrical and these surfaces preferably fit closely together to form a seal.

Therefore, the application arrangement piece with a through-hole provides several functions: it guides and receives the tip of the dispenser so that the dispenser is accurately fitted into said through-hole. Thus, the tip of the dispenser engages in the through-hole of the adapter to provide a detachable but fluid-tight seal. However, it is not necessary that the tip extends through the whole application arrangement piece, it is sufficient that the tip extends in part into the piece. Furthermore, the adapter serves as a reducer which decreases the provided opening of the container to an opening wherein the dispenser fits in. Therefore, the adapter establishes the fluid communication between the interior of the container and the chamber of the dispenser. It facilitates the rapid and safe access to the contents produced, contained and stored within the container.

The adapter is generally made of plastic material which may readily be used in the pharmaceutical sector. Examples thereof are rubber, particularly natural or synthetic rubber, plastic materials such as elastomeric or thermoplastic materials or thermoplastic elastomers such as polyisoprene. The material may have a coating in order to create a better barrier against leakage of the liquids.

The adapter is readily available and may be purchased or can be manufactured reliably and at low cost. For example, the adapter may be stamped from a plastic sheet material in a mass production.

### Component E)

Finally, the kit-of-parts contains instructions (component E)) for use of the kit which allow to readily use and handle the components A) to D) of the kit-of parts.

Therefore, the kit-of-parts of the present invention is directed to a combination of components that permits to achieve a stable, homogeneous liquid formulation which enables to facilitate the dose adjustment, provide an accurate dosage and simplify and enhance the comfort of the treatment and/or prevention of diseases such as heart diseases in a patient/animal patient.

The present invention is also directed to the kit-of-parts, wherein the instructions of component E) comprise the following:

i) adding the whole content of the packaging into the container;
ii) shaking the container; and
iii) removing the desired volume from the container.

According to a preferred embodiment the present invention is directed to a kit-of-parts, wherein the instructions of component E) comprise or consist of the following:
a) opening of the container;
b) opening of the packaging;
c) adding the whole content of the packaging into the container;
d) applying the adapter, having an application arrangement piece with a through-hole onto the container;
e) closing the container;
f) shaking the container;
g) opening the container;
h) inserting the tip of a dispenser into the through-hole of the application arrangement piece of the adapter;
i) removing liquid from the container, using the dispenser;
j) disconnecting the dispenser from the adapter;
k) administering the liquid with/by means of the dispenser to the patient/animal or to patient/animal food;
l) closing the container;
m) optionally repeating instructions f) to l);
   whereby the sequence of instruction a) and instruction b) may also be reversed,
   whereby the sequence of instruction k) and instruction 1) may also be reversed.

In the following the instructions of component E) of the kit-of-parts according to the present invention which are equivalent to the steps of the method for administering a pharmaceutically active ingredient in the form of solid coated one or multi-layered particles or pellets according to the present invention will be described in detail.

In instruction or step a) the opening of container B) comprising a liquid oily composition to disperse or suspend the solid coated one or multi-layered particles contained in packaging A) is performed. The container is preferably a glass vial or glass bottle. Preferably the container has a screw cap which is removed.

In another embodiment of the present invention container B) is packed into a tight pouch. In this case the pouch needs to be opened and removed before starting with step a).

In another embodiment of the present invention a seal can optionally be added to the mouth of the container B) which needs to be removed after step a) before continuing.

Subsequently in step b) packaging A) containing a defined amount of a pharmaceutically active ingredient in the form of solid coated one or multi-layered particles or pellets is opened. Depending from the type of packaging selected it may be pulled or torn open or cut to be opened.

As a matter of course the sequence of instructions or steps a) and b) may also be reversed.

In instruction or step c) the whole content of packaging A) that is the solid coated one or multi-layered particles or pellets which are optionally taste masked containing a pharmaceutically active ingredient is transferred into the container B). Container B) contains dispersing or suspending carrier(s) for the formulation, i.e. an oily composition as already described and optionally further additives. Thus, the liquid dosage form to be administered to the patient/animal is obtained.

The content and the amount of the packaging A) and the content and the amount of container B) are selected in such a manner that the obtained liquid dosage form (solid coated one or multi-layered particles, optionally taste masked, +oily composition) provides the pharmaceutically active ingredient in the desired concentration to be administered to the patient/animal.

In instruction or step d) an adapter D) having an application arrangement piece with a trough-hole is applied onto the opening and mouthpiece of container B). The adapter is designed such as to engage with the opening and mouthpiece of the container in order to seal the inner walls of the container in a fluid-tight manner. The through-hole of the application arrangement piece is adapted to the form of the tip of the dispenser C) which may be a syringe-like dispenser. Preferably the adapter has the form of or is a usually employed adapter of a dosing pipette.

In instruction or step e) the container is again closed by using the sealant which was originally present on the container prior to opening. The sealant may be a screw cap or another type of sealant which is designed and dimensioned to accommodate the container having applied the adapter thereon. Therefore, the container, adapter and original sealing of the container may be constructed in such a manner that the container on which the adapter is already applied may be again sealed with the original sealing. Therefore, it is not necessary to remove the adapter from the container in order to seal again the container with the original sealing.

In another embodiment of the present invention the sealant may consist of an additional seal layer which is either a permanent part of the sealing or added to the mouth of the bottle during the storage period of container B) before first use.

The sealant or sealing of the container is construed to seal the container in a fluid-tight manner. To this purpose the sealing may contain an additional gasket seal designed and dimensioned to accommodate also the adapter.

In instruction or step f) the container is shaken by hand in order to provide the stable and homogeneous mixture of the obtained liquid dosage form (solid coated one or multi-layered particles, optionally taste masked, +oily composition). For example, the shaking may be performed for several seconds, preferably 30 seconds in order to arrive at a homogeneous distribution of the solid coated one or multi-layered particles in the oily matrix. The shaking may be repeated. The obtained mixture is considered to be homogeneous if the achieved mixture is uniform in composition, i.e. the solid coated one or multi-layered particles, optionally taste masked, are distributed uniformly in the liquid matrix.

According to a preferred embodiment the liquid oily composition and likewise the obtained dispersion, particularly suspension, preferably show a high viscosity. In a preferred embodiment the oily composition has a thixotropic property so that the viscosity is lowered due to a shaking. This behavior (thixotropy) can for example be reached by the presence of the mentioned mixture of colloidal silica, such as hydrophilic colloidal silica, and hydrophobic colloidal silica. Thus, the shaking of the oily composition or the obtained suspension assists to improve the flow characteristics thereof so that it can be easily applied via an oral dispenser.

After instruction or step g) and opening the container again in instruction or step h) the tip of a dispenser C) is inserted into the through-hole of the application arrangement piece of the adapter D) in order to provide a fluid communication between the chamber of the dispenser C) and the content of the container B). The exterior surface of the tip of the dispenser and the interior surface of the through-hole of the application arrangement piece of the adapter engages with each other in such a manner that these surfaces preferably fit closely together to form a seal.

In instruction or step i) the desired amount of liquid dosage form is removed from container B) using the dispenser. A correlation between a scale present on the dispenser and the weight of the patient/animal patient to be treated may facilitate the dose to be administered to the patient/animal. In order to extract the liquid dosage form from the container B) the container is preferably held upside down with the opening of the container in direction downwards.

According to instruction or step j) the dispenser is disconnected from the adapter and in instruction or step k) the liquid is administered with/by means of the dispenser to the patient/animal or to patient/animal food. The drug may be released directly into the mouth of the patient/animal or may be injected or put into a meal of the patient/animal patient.

Subsequently the container is closed again in instruction or step l), preferably directly after withdrawal of the liquid dosage form, in order to protect the liquid dosage form against contamination or incoming moisture. As a matter of course the sequence of instructions or steps k) and 1) may also be reversed.

According to a preferred embodiment of the present invention the instructions or steps e) to j) may be repeated, preferably for a number of times (instruction or step m)). Thus, practically the whole content of the obtained liquid dosage form which is sufficiently stable and can be maintained in a homogeneous form may be used

Therefore, the dispenser may be used several times in order to uptake the desired amount of liquid dosage form in the defined concentration for several treatments of one or more patients/animal patients. It is particularly preferred to shake the container B) containing the solid coated one or multi-layered particles or pellets, which may be taste masked, and the oily composition prior to any use in order to secure that a homogeneous mixture is still obtained and if a thixotropic composition is provided in order to establish better flow characteristics.

If instruction or step f) is repeated, the shaking of the obtained dispersion or suspension may be shortened to some seconds, such as 5 to 10 seconds.

According to one embodiment, in case the pharmaceutically active ingredient is an I_{F}-channel inhibitor, the liquid pharmaceutical dosage form contained in the container B) preferably comprises the pharmaceutically active ingredient in a final concentration of 0.1 to 20 mg/ml, preferred 0.5 to 5 mg/ml, more preferred 0.5 to 4 mg/ml, and most preferred 0.5 to 3 mg/ml. This has turned out to be useful for the intended therapy. In fact, the appropriate concentration of each pharmaceutically active ingredient used can be adjusted and adapted with respect to the intended treatment and the patient or patient group to be treated.

After the administration has taken place, the dispenser may be cleaned with a dry cloth and preferably sealed with a suitable sealant such as a cap in order to avoid contamination or entry of moisture into the dispenser.

Taking components A) to E) of the kit-of-parts as well as the above instructions or steps a) to m) of the kit-of-parts into account, the advantages of the kit-of-parts are manifold:

The user may at home prepare and obtain the desired liquid dosage form in a ready to use form based on a low number of steps in an easily manageable manner. The provided liquid dosage form may be used several times for a patient/animal or different patients/different animals. The ready to use liquid dosage form is storage stable over an extended period of time and allows an accurate dose adjustment for each and every case. The obtained and reproducible homogeneity of the liquid dosage form assures that the accurate dosage may be administered. Further, the administration system in form of the kit-of parts is provided without the necessity of using a syringe with a sharp needle.

The dispenser, such as a syringe-like dispenser, preferably provides a scale on the surface which allows to easily correlate patient's/animal's weight and the dose to be administered. Furthermore, the oral dispenser may be used to provide the respective dose directly to the patient/animal to be treated, i.e. into the mouth of the patient/animal or the food of the patient/animal. In this connection the ease of administration also contributes to the quality of life of the patient/animal in need of such treatment as the treatment can only be really successful when it is possible to administer the pharmaceutical composition for the treatment and/or prevention of diseases to the patient/animal such as feline, preferably cat. In addition, the treatment and/or prevention is less time consuming for the keeper or owner of an animal, because complicated steps of finding out and determining the exact dose for each animal to be treated may be omitted. The present invention will improve the animal owner's compliance and therefore the safety of the drug product due to ease of administration.

The invention also relates to a kit-of-parts for the use in a method of treating and/or preventing a heart disease in a mammalian patient, preferably in a companion animal, more preferred in a feline, in particular in a cat.

Known heart diseases include for example coronary heart diseases, cardiomyopathies, cardiovascular diseases, heart failure, hypertensive heart diseases or valvular heart diseases. For example, feline heart failure is predominantly caused by different cardiomyopathies. The most common feline cardiomyopathy (CMP) is hypertrophic cardiomyopathy (HCM), followed by restrictive cardiomyopathy (RCM), unclassified cardiomyopathy (UCM), dilated cardiomyopathy (DCM) and arrhythmogenic right ventricular cardiomyopathy (ARVC).

Typically cats with underlying cardiomyopathies remain clinically asymptomatic in the early stages of the disease until they are presented to the veterinarian because the disease has progressed and ventricular diastolic and/or systolic function is severely impaired resulting in heart failure. Cats at this stage present either mild to moderate clinical symptoms with tachypnea up to severe respiratory distress (dyspnea) due to pulmonary edema and/or pleural effusion requiring emergency treatment with oxygen therapy and intravenous diuretic and thoracocentesis in case of pleural effusion.

The term “heart failure” as used herein relates to a condition in which a problem with the structure or function of the heart impairs its ability to supply sufficient blood flow to meet the body's needs, in particular any contractile disorder or disease of the heart. Clinical manifestations are as a rule the results of changes to the heart's cellular and molecular components and to mediators that drive homeostatic control. Heart failure is caused by different diseases of the heart. There are several main indications with different etiology that may result in heart failure such as hypertrophic cardiomyopathy (HCM), dilated cardiomyopathy (DCM), restricted cardiomyopathy (RCM), unspecified cardiomyopathy (UCM) and arrhythmogenic right ventricular cardiomyopathy (ARVC). Preferably these cardiomyopathies are classified according to Kittleson (Kittleson, Textbook of veterinary internal medicine, Eds. Ettinger, Feldman, Elsevier Saunders, Philadelphia. 2005, 1082).

The term “patient” as used herein relates to a patient, particularly a mammalian patient, preferably a companion animal, more preferred a feline, in particular a cat, even more preferred domestic cats, in particular to feline suffering from a heart disease, particularly heart failure, even more preferred to cats suffering from a heart disease, particularly heart failure.

According to an embodiment of the invention the kit-of-parts is intended for the use in a method of treating and/or preventing feline patients suffering from heart failure due to one or more of the following etiologies HCM, DCM, RCM, UCM and/or ARVC.

In the following a preferred embodiment of solid coated multi-layered particles or pellets which are optionally or preferably taste masked, contained in package A) will be described in detail.

According to a preferred embodiment the present invention is directed to a kit-of-parts, wherein the solid coated multi-layered particles or pellets of A) comprise or consist of:
a) an inert core,
b) one or more coating layer(s), comprising or consisting of a pharmaceutically active ingredient and a binder,
c) an intermediate coating layer free from a low molecular weight water-soluble ionic compound, preferably comprising or consisting of a water-soluble pharmaceutical film-forming compound in form of poly(1-vinylpyrrolidin-2-one) (PVP), and
d) an outer coating layer free from a low molecular weight water-soluble ionic compound, preferably comprising or consisting of a mixture comprising or consisting of 60-90% (w/w) ethylcellulose (EC) and 10-40% (w/w) hydroxypropyl methylcellulose (HPMC).

It shall be noted that as used herein, the singular forms “a”, “an”, and “the” include plural reference unless the context clearly dictates otherwise. Thus, for example, reference to “a particle” includes a plurality thereof.

The multi-layered particles or pellets of this embodiment are well-assembled coated drug loaded subunits. They can be produced starting from inert particles or spherical carriers that are coated stepwise by specific layers. For this purpose they are first layered once or more than once with a layer comprising the specific pharmaceutically active ingredient; the drug layer is then covered by an intermediate coating layer (for example a seal coating) and then by an outer coating layer (for example final coating or taste masking coating).

A taste masking coating may be present in case the pharmaceutically active ingredient is bitter tasting and highly water-soluble such as the I_{F}-channel inhibitors, especially in its salt form. For example animals, particularly cats, have the habit to break down their food by biting on it several times thereby destroying the dosage form contained and release the unpleasant tasting drug. Further, if the pharmaceutically active ingredient present is water soluble the phenomenon occurs that at least traces of the compound diffuse within the particle and/or the coating layers already during the coating process itself, i.e. before the respective coating layer has dried, just because the compound is solved at least partially by the solvent of the respective coating layer material during the coating process. Accordingly, whereas the main portion of the compound might be hidden away by the established coating techniques, traces of it might still be perceivable by the patient which is often not acceptable for a pharmaceutical composition for the treatment of patients/animals which consequently refrain from taking it.

The multi-layered particles or pellets used according to this embodiment—if a taste masking is present—overcome these deficiencies. In the multi-layered particles or pellets the masking of the taste is so efficient that even taste sensitive animals like cats or dogs as patient population do not refrain from the voluntary ingestion of the fully formulated pharmaceutical composition.

The particles are “multi-layered” which means that they are characterized by a well-designed architecture in which inert particles or spherical carriers are coated by one or more layers comprising the specific pharmaceutically active ingredient, on top of this by an intermediate coating layer (e.g. seal coating) and then by an outer coating layer (e.g. final or taste masking coating). Within the context of the present invention the multi-layered particles are used synonymously for pellets as already explained. Details of this architecture as well as materials that can be used for it are explained below.

The architecture of the multi-layered particles or pellets, comprising an inert core and the coating layers, will be described in the following in detail.

### a) Inert Core

The “inert core” according to this embodiment is a particle or spherical carrier. It has been found advantageous to use particles with a diameter of about 50 to 300 μm, preferably with a small degree of variation. Preferred sub-ranges are defined below.

The material used for the inert core is chemically inert, in the sense that it does not react with any of the other ingredients of the multi-layered particle and especially does not interfere with the intended pharmacological mechanism exerted by the pharmaceutically active ingredient of the multi-layered particle. Examples for a material used for the inert core are cellulose, especially microcrystalline cellulose, starch, lactose, sugar, mannitol or mixtures thereof.

The coating process as regularly starts with the material for the inert cores. It is intended that each single particle of this starting material serves as a single core for each multi-layered particle. The process is a statistical process with Gauss' distribution curves for the results of each step. This leads to statistical results, meaning that each layer has got an average thickness. It is preferred that multi-layered particles or pellets with more than only one single inert core particle be sieved off and in doing so be excluded from incorporation into a pharmaceutical composition.

The methods for the coating process, i.e. for the subsequent addition of layers onto a smaller particle are per se known to the person skilled in the art. Such are described for example in the textbook “Developing Solid Oral Dosage Forms—Pharmaceutical Theory and Practice”, chapter 34, edited by Yihong Qiu, Yisheng Chen and Geoff G. Z. Zhang; Elsevier (2009). Also the apparatus for granulation processes are known to the person skilled in the art, for example “Developing Solid Oral Dosage Forms—Pharmaceutical Theory and Practice”, chapter 34, edited by Yihong Qiu, Yisheng Chen and Geoff G. Z. Zhang; Elsevier (2009).

### b) One or More Coating Layer(s)

The one or more coating layer(s) comprising or consisting of a pharmaceutically active ingredient and a binder, which is layered onto the core particle, contains the pharmaceutically active ingredient, regularly in its chemical form appropriate for the medical treatment. It further contains a binder and optionally other ingredients that as a mixture are intended to form the respective coating layer.

Useful binder materials include but are not limited to tragacanth, gelatin, starch, cellulose materials such as methyl cellulose, microcrystalline cellulose and sodium carboxy methyl cellulose, alginic acids and salts thereof, polyethylene glycol, PVP, guar gum, polysaccharide acids, sugars, invert sugars and the like. Preferred are hydroxypropyl methylcellulose (HPMC) and/or polyvinylpyrrolidone (PVP).

The material HPMC (also called Hypromellose; the name HPMC according to DIN EN ISO 1043-1: 2002-06, E 464) is a mixed ether of cellulose with 2-hydroxypropyl and methyl groups. Technically it is usually prepared by the reaction of cellulose with methylchloride and propylene oxide. It can be purchased commercially, e.g. by the supplier Harke Pharma under the trade name PHARMACOAT®.

The material PVP (also called Poly(1-vinylpyrrolidin-2-one) or Polyvidon; the name PVP according to DIN EN ISO 1043-1: 2002-06) is a vinylpolymer of the general structural formula:

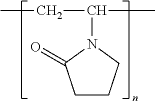

and with molecular weights between 2,500 and 750,000 g/mol, which again depends on the degree of polymerization. They are characterized by K values which are calculated from the viscosity of 1% or 5% aqueous solution. PVPs are technically usually prepared by radical polymerization of 1-vinylpyrrolidin-2-one.

Binders may be used in an amount of up to 60% (w/w). It has been found advantageous that the drug layer on the pellets consists of up to 40% (w/w), preferably 5 to 20% (w/w) of binder. More preferred ranges are defined below. In another preferred aspect according to this embodiment the drug layer on the pellets consists of 1-30% (w/w) of the binder.

The coating material for this layer can advantageously be applied in the form of a liquid solution or suspension of the binder material in water which is allowed to evaporate after the addition of the material.

In this embodiment the pharmaceutically active ingredient may be selected from the class of I_{F}-channel blockers as already described. Particularly preferred is cilobradine or a pharmaceutically acceptable salt thereof such as cilobradine hydrochloride.

While using the fluid bed coating process, it is preferred to spray the liquid mixture onto the core particles. It is preferred that the coating layer comprising the pharmaceutically active ingredient and a binder is added up to a thickness of more than 20 μm, and increasingly preferred more than 30, 40, 50, 60, 70, 80 and 100 μm and mostly preferred between 50 and 100 μm. Such variability allows adding the pharmaceutically active ingredient to an extent that is appropriate to reach the desired concentration of active ingredient in the finally prepared solid taste masked formulation.

This coating step can be repeated in order to allow the addition of (a) second coating layer(s) comprising the pharmaceutically active ingredient. The material for this optional second layer can be identical with the first one. However, variations of this composition in order to optimize physicochemical parameters also lie well within the ambit of the invention. The second layer can be added to the same or to a different thickness than the first layer. In doing so a double coating layer comprising the pharmaceutically active ingredient of more than 200 μm in total is possible.

### c) Intermediate Coating Layer

The feature that the intermediate coating layer preferably comprises or consists of a water-soluble pharmaceutical film-forming compound is to be understood as a complete coating layer around/on top of the layer comprising the pharmaceutically active ingredient serving especially two purposes: (i) mechanical stability for the particle as a whole and especially for the integrity of the layer comprising the pharmaceutically active ingredient, and (ii) being a barrier against the dissolution of the pharmaceutically active ingredient within the particle, especially during the production of the particle as well as during the storage period of the complete multi-layered particles or pellets and the fully formulated pharmaceutical composition. Besides the water-soluble pharmaceutical film-forming compound, further ingredients can optionally be present in this coating layer.

According to this embodiment, this coating layer is layered onto the one or more coating layer(s) comprising the pharmaceutically active ingredient and a binder. It has been found advantageous to apply this coating layer by way of suspension or solution in water or an organic solvent, also mixtures of water and e.g. ethanol are possible.

An appropriate water-soluble film-forming compound is poly(1-vinylpyrrolidin-2-one) (PVP).

In a preferred mode the material for this coating layer additionally comprises talc and/or magnesium stearate. In another aspect this coating layer additionally comprises one or more of the following anti-tacking agents such as talc, magnesium stearate, calcium docosanoate, stearic acid, calcium arachinate, hydrogenated castor oil, or triglycerides.

The mixture for this coating layer can advantageously be added to the respective particles in the form of an aqueous solution or suspension. Employing an aqueous process is especially advantageous with respect to cost, work safety and environmental reasons.

According to this embodiment it is preferred to add PVP and optionally talc in the form of a solution in an organic solvent like ethanol and/or acetone.

The coating layer (c) is preferably a seal coating.

The thickness of the coating layer (c) can vary with respect to the specific particle. Preferably, it lies between 5 and 40% (w/w) of the drug layered particles, more preferred between 10 and 30% (w/w) even more preferred between 15 and 25% (w/w).

The ideal thickness in each case can be developed experimentally by a person skilled in the art, with respect to the following considerations: The coating, preferably seal coating, serves as protective coating to mechanically stabilize the complete particle and the pharmaceutically active ingredient containing layer for the following coating steps and to prevent migration of the drug active ingredient into the outer layer when the final coating is applied or even during storage. The coating, preferably seal coating is deemed to be thick enough when no (or only traces of) the compound can be detected on the outer surface of the particles. An appropriate detection method is Energy Dispersive X-ray analysis (EDS).

### d) Outer Coating Layer

The features that the outer coating layer preferably comprises or consists of (i) a mixture comprising 60-90% (w/w) ethylcellulose (EC) and 10-40% (w/w) HPMC is a complete coating layer around/on top of the intermediate coating layer (preferably seal coating). Without wishing to be bound by this theory, it is believed that especially this coating is responsible for the good taste masking properties of the multi-layered particles or pellets according to this embodiment, if a taste masking is intended, especially by inhibiting the diffusion of the pharmaceutically active ingredient to the surface of the particle, neither during production nor during the storage of the particles and/or the derived pharmaceutical compositions. It further serves the purpose of mechanical stabilization of the particle as a whole and especially for the integrity of the layers underneath.

A mixture comprising or consisting of 60-90% (w/w) ethylcellulose (EC) and 10-40% (w/w) HPMC is preferably used as material for the outer coating layer.

The material ethylcellulose (EC; the name according to DIN EN ISO 1043-1: 2002-06) is the ether of cellulose with ethyl groups. Technically it is usually prepared by the reaction of alkalicellulose with ethylchloride, which can lead to types of EC differing with respect to the degree of substitution. Ethylcelluloses with substitution degrees between about 1.1 and 1.4 are soluble in water; those with higher degrees of substitution are soluble in organic solvents. Commercially available EC types with substitution degrees between about 2.2 and 2.6 are thermoplastic with a softening point between around 150 and 160° C. Commercially available EC types are offered with different molecular weights which further influence the physico-chemical properties of the material like their viscosity.

According to an embodiment it is preferred to use EC with a substitution degree between 2.2 and 2.6 and/or with a degree of polymerization resulting in a viscosity of 41-49 mPa·s (Millipascal Second) (measured as 5% solution in 80% toluene and 20% ethanol). Such EC can be purchased commercially, e.g. by the supplier Dow Chemical under the trade name ETHOCEL® Std. 45.

The material HPMC has already been presented above.

According to an embodiment, the ratio of HPMC and EC can be varied in the given frame in order to achieve the desired release profile, especially with respect to the following considerations. Without wishing to be bound by this theory it is assumed that in a blend of EC and HPMC according to this embodiment, the HPMC dissolves first after ingestion by the patient and thus leaves pores in the EC film. Then water can penetrate through the pores into the core of the particles and thereby allow the pharmaceutically active ingredient to be released with a lag time, i.e. the time the HPMC needs to be washed out of the film. The EC/HPMC film composition and the coating thickness (see below) can be optimized in a way to generate a lag time that is considered long enough to allow the dosage form to be ingested without the drug being released.

With regard to the EC/HPMC films, the coating thickness and the way the coating is applied to the particles can be varied/optimized in order to achieve the desired drug release profile or make the process feasible, i.e. aqueous vs. organic solvent spraying process. Such variations lie well within the reach of a person skilled in the art. Preferred values are disclosed in more detail below.

Moreover, the final coating layer may advantageously comprise further substances. For example magnesium stearate is used to counteract electrostatic charging during processing. Another example is silicon dioxide to reduce electrostatic charging of the product. Further examples are talc, magnesium stearate, calcium docosanoate, stearic acid, calcium arachinate, hydrogenated castor oil, or triglycerides.

The outer coating layer (d) may be preferably in form of a final or taste masking coating.

Both, the intermediate coating layer (preferably seal coating) (c) and the outer coating layer (preferably final or taste masking coating) (d) are characterized by the feature “free from a low molecular weight water-soluble ionic compound”.

Without wishing to be bound by this theory, this feature has been found to be one important reason for the efficiency of the taste-masking of the particle as well as of the derived pharmaceutical compositions and thus the usefulness of this embodiment, in case a taste masking is intended. Whereas the state of the art teaches that compounds like NaH₂PO₄, Na₂HPO₄, Na₂CO₃ or citrates, especially the salts of strong acids with strong bases, are useful as dissolution aids of the particles and thus helpful for the application of the pharmaceutical ingredient to the patient's body. However, in the context of the taste-masking of a pharmaceutically active ingredient which is water-soluble and comprises a bitter taste, it was found to be advantageous to avoid such compounds.

However, less ionic compounds like talc, stearic acid, sodium lauryl sulfate or magnesium stearate, especially oxides or the salts of weak acids used e.g. as lubricants, coloring agents and/or anti-tacking agents and the like are found to be not so critical for the taste-masking effect and are thus allowed as optional ingredients. This aspect will be explained in more detail below.

The selection of such additional compounds in the layers (c) and (d) is thus limited by their ability to dissolve in water. They are uncritical and therefore not water-soluble ionic compounds in the sense of the application as long as 1 g of such a compound is completely dissolved by not less than 100 ml water, at a temperature of 25° C. and 1013.25 hPa atmospheric pressure. Increasingly preferred they possess solubility values of at least 250, 500, 750, 1000, 2000, 3000, 4000, 5000, 6000, 7000, 8000, 9000, 10000, 15000 and more than 20000 ml water for the total dissolution of 1 g of the respective substance under the same conditions.

Compounds with low molecular weight, according to this embodiment, are defined as those with a molecular weight below 750 Da, increasingly preferred below 700, 600, 500, 400, 300, 200 and 150 Da.

The different coating materials are preferably applied to the respective particles in the form of a liquid solution or suspension in an appropriate solvent. This solvent can be selected from (purified) water or organic solvents.

One preferred mode is a multi-layered particle or pellet, which is optionally taste masked, further comprising one or more additional layers between the inert core and/or between one of the coating layers (b) to (d) and/or on top of coating layer (d).

Such additional layers can be coating layers in the sense of the present invention, i.e. making up a complete coat or closed film around the particles underneath, or just a layer of additional material between or on top of the other coating layers.

For example one of the coating layers disclosed herein can be applied more than once. Further, e.g. protective layers might be concluded from the state of the art.

In an alternative, a material is added that does not form a closed film but still exerts positive effects. These materials can for example be anti-tacking agents that ameliorate the further processing of the respective particulate material. Such anti-tacking agents are per se known for the skilled person. They can for example further consist of pigments, and/or flavors which advantageously be added on top of the outer coating layer (final or taste masking coating). Also such materials are per se known for the skilled person.

One preferred mode is a multi-layered particle or pellet, wherein the material for the inert core is cellulose, preferably microcrystalline cellulose.

As explained above, the chemically inert material used for the inert core can be selected from different materials. A preferred material is cellulose, especially microcrystalline cellulose. Especially this material has turned out to be useful, with respect to the further processing of the material, but also with respect to the physical properties of the finally formulated multi-layered particles. Particles of microcrystalline cellulose can be purchased e.g. under the trade name CELLETS® 100 from the company Syntapharm (Harke Group), Mulheim, Germany.

In another preferred aspect the inert core particles are preferably selected from a pharmaceutically acceptable material which upon contact with water shows a minimal or negligible swelling. Preferable materials are selected from lactose, carbohydrates, sugar alcohols, such as mannitol, sorbitol, maltitol, glucose, non-pareil-seeds, calcium phosphate, cellulose, preferably microcrystalline cellulose (MCC), and starch, and mixtures thereof, more preferably lactose, most preferably agglomerated alpha-lactose-monohydrate [Ph.Eur./USP-NF/JP] with a particle size d₅₀ of about 180 μm.

Lactose such as agglomerated lactose, with the characteristics described above, is also suitable for use in the core because of its particle size and non-hygroscopicity.

One preferred mode is a multi-layered particle or pellet, wherein the binder for coating layer (b) comprising the pharmaceutically active ingredient is selected from HPMC or PVP or a mixture thereof and optionally additional ingredients, preferably the HPMC Hypromellose, USP Substitution Type 2910 (apparent viscosity 4.8-7.2 mPas) and/or PVP K30 and optionally additional ingredients.

Such materials have already been described above. A preferred material for this coating layer (HPMC Hypromellose, USP Substitution Type 2910; apparent viscosity 4.8-7.2 mPas) is commercially available under the trade name PHARMACOAT® 606, sold by Harke Pharma GmbH, Mulheim, Germany.

Alternatively preferred is the material PVP K30 with a molecular weight between 44 and 54 kg/mol. Such PVP can be purchased commercially, e.g. by the supplier BASF, Ludwigshafen, Germany, under the trade name KOLLIDON® 30.

One preferred mode is a multi-layered particle or pellet, wherein coating layer (b) comprises or consists of 60 to 70% (w/w) of the pharmaceutically active ingredient, 25 to 35% (w/w) of HPMC as binder and 0.5-3% (w/w) magnesium stearate.

Especially in this context magnesium stearate is an advantageous lubricant with a positive influence especially on the coating process. An appropriate magnesium stearate is for example PARTECK® LUB MST, sold by the commercial provider Merck KGaA, Darmstadt, Germany.

One preferred mode is a multi-layered particle or pellet, wherein the intermediate coating layer (preferably seal coating) (c) additionally comprises talc, preferably 10 to 30% (w/w) talc, more preferred 15 to 25% (w/w) talc, most preferred 20-25% (w/w) talc.

In another preferred aspect the said intermediate coating layer (preferably seal coating) (c) additionally comprises preferably 5 to 30% (w/w) talc.

One preferred mode is a multi-layered particle or pellet, wherein the material for the intermediate coating layer (c), PVP, is selected from PVP K 30.

One preferred mode is a multi-layered particle or pellet, wherein the material for the intermediate coating layer (c), comprises or consists of 70-80% (w/w) PVP and 20-25% (w/w) talc. Especially this mixture has proven to be useful.

In another preferred aspect said intermediate coating layer (c), comprises or consists of 70-95% (w/w) PVP and 5-30% (w/w) talc.

One preferred mode is a multi-layered particle or pellet, wherein the material for the outer coating layer (preferably final or taste masking coating) (d) is selected from EC with a viscosity range of 41-49 mPas (measured as 5% solution in a mixture of 80% toluene and 20% ethanol and an ethoxyl content of 48.0-49.5%) and/or HPMC with a methoxyl content of 28-30%, a hydroxypropyl content of 7-12% and a viscosity range of 4-6 mPas (measured as 2% solution in water). Also this alternative taste masking coating with these selected materials is particular useful.

An appropriate EC (ethylcellulose) is for example the product ETHOCEL® 45 cps STD Premium, sold by the commercial provider Dow Chemicals, Schwalbach, Germany. An appropriate magnesium stearate for this taste masking coating is for example PARTECK® LUB MST, sold by the commercial provider Merck KGaA, Darmstadt, Germany.

One preferred mode is a multi-layered particle or pellet, wherein the material for the outer coating layer (d), comprises or consists of 50-75% (w/w) EC, 15-40% (w/w) HPMC and O-25% (w/w) magnesium stearate, preferably 50-60% (w/w) EC, 20-25% (w/w) HPMC and 17.5-22.5% (w/w) magnesium stearate.

For examples of such coatings it is referred to WO 2013/024023 (PCT/EP2012/065680) the whole disclosure thereof being incorporated in the present disclosure by reference.

A preferred mode of this aspect is made up by multi-layered particles or pellets, wherein the material for the outer coating layer (d) makes up a coating level of at least 25% (w/w) (based on the weight of the particles beneath this coating), preferably 25 to 100% (w/w), more preferred 50 to 90% (w/w), even more preferred 70 to 80% (w/w), most preferred 72.5 to 77.5% (w/w).

One preferred mode of this aspect is a multi-layered particle or pellet, wherein the outer coating layer (d) of the particle is characterized (mixture comprising EC and HPMC) by a thickness of 10 to 150 μm, preferably 12 to 120 μm, more preferred 15 to 100 μm, most preferred 20 to 50 μm. All of these values are to be understood with a variance of ±10 μm.

An outer coating is obtainable by a final coating step using an aqueous coating solution and a coating level of 200% with a variance of ±10 μm. Such particles show an advantageous taste-masking effect accompanied with an advantageous dissolution profile and are therefore preferred embodiments. Variations of the process, especially the use of an organic solvent and/or different coating levels can be applied by a person skilled in the art in order to reach the other named values of thickness of the respective outer coating layers (d). Such particles are equally preferred.

As said above, multi-layered particles or pellets can comprise additional layers that can be coating layers in the sense of this invention, i.e. making up a complete coat or closed film around the particles underneath, or just a complete or incomplete layer of additional material between or on top of the other coating layers.

One preferred mode of this aspect pertains to multi-layered particles or pellets, wherein the one or more additional layers between the inert core and/or between one of the coating layers (b) to (d) and/or on top of coating layer (d) comprise(s) colloidal silicon dioxide (silica), preferably 0.1-5% (w/w) (based on the weight of the final particles), more preferred 0.2-2.5% (w/w), most preferred 0.2-1% (w/w).

It has been found advantageous to add such material on top of the particles or pellets during the production process after drying of one coating layer and before the addition of the next coating layer, especially as anti-tacking agent.

One preferred mode of this aspect pertains to multi-layered particles or pellets according to this embodiment, wherein an additional coating layer(s) is a final outer-coating on top of coating layer (d) and the material for this additional coating layer(s) is selected from HPMC with a methoxyl content of 28-30%, a hydroxypropyl content of 7-12% and a viscosity range of 4-6 mPas (measured as 2% solution in water).

This has been found to be very advantageous for the multi-layered particles or pellets that are intended for the incorporation into overall liquid formulations, especially oily liquid pharmaceutical compositions. The additional layer serves two purposes: (i) a mechanical protection against the other particles in the suspension and (ii) an additional protection against water that might tend to diffuse through the oily suspension into the particles.

One preferred mode is a multi-layered particle or pellet, wherein one or more of the layer material(s) comprise(s) additional substances, preferably filler substances, binders, wetting agents, glidants, lubricants, dispersing agents, coloring agents and/or anti-tacking agents, selected from one or more of: mannitol, starch, talc, titanium dioxide, sodium lauryl sulfate, sodium dodecylsulfate, stearic acid, magnesium stearate, silica and medium chain triglycerides.

In addition, preferred multi-layered particles or pellets are those, wherein one or more of the layer material(s), preferably for one of the coating layer (c) and/or coating layer (d) comprise(s) additional substances selected from flavoring agents, pigments and substances for the reduction of electrostatic charging, more preferred meat flavor, pigments and/or silicon dioxide.

For example, it has been found advantageous to choose an anti-tacking agent, preferably magnesium stearate, e.g. magnesium stearate PARTECK® LUB MST, commercially available, e.g. by the supplier Merck KGaA, Darmstadt, Germany, as an additional ingredient for coating layer (b). The optionally incorporated anti-tacking material can advantageously be added in the form of a suspension in a solvent, e.g. suspended in the solution/suspension used for the adding of the respective coating layer during the respective coating step. Alternatively it can be added in the form of a powder onto the dried particles after the respective layer is built up and dried.

In another/additional preferred mode the material for the intermediate coating layer (c) additionally comprises talc and/or magnesium stearate.

Coloring agents may include but are not limited to titanium dioxide, and dyes suitable for food and natural coloring agents such as grape skin extract, beet red powder, beta-carotene, annato, carmine, turmeric, paprika, etc. The amount of coloring agent used may range from about 0.05 to about 3.5% (w/w) of the total dosage form.

However, as discussed above, the feature “free from a low molecular weight water-soluble ionic compound” is limiting. The selection of such compounds is limited by their ability to dissolve in water, as explained above.

Further, not less preferred embodiments pertain to multi-layered particles or pellets, wherein:
the inert core is characterized by a diameter of 50 to 300 μm, preferably 75 to 250 μm, more preferred 100 to 200 μm;
wherein the final coated particle is characterized by an overall diameter of 80 to 800 μm, preferably 90 to 600 μm, more preferred 100 to 400 μm; and/or
wherein the pharmaceutically active ingredient in its incorporated chemical form makes up 1 to 50% (w/w) of the final multi-layered particle, preferably 2 to 25% (w/w), more preferred 3 to 22.5% (w/w) and most preferred 5 to 20% (w/w).

The multi-layered particles according to this embodiment allow an effective taste masking of a pharmaceutically active ingredient having a bitter taste such as an I_{F}-channel inhibitor, accompanied by a useful dissolution profile in the patient's mouth or intestine.

One particularly preferred mode of the present invention is a kit-of-parts,

wherein the solid coated multi-layered particles or pellets comprise or consist of:
a) an inert core, comprising or consisting of microcrystalline cellulose,
b) one coating layer comprising or consisting of 60 to 70% (w/w) of the pharmaceutically active ingredient, 25 to 35% (w/w) of HPMC as binder and 0.53% (w/w) magnesium stearate,
c) an intermediate coating layer (preferably seal coating), comprising or consisting of 70-80% (w/w) PVP K 30, 20-25% (w/w) talc and 0.5-5% (w/w) silicon dioxide and
d) an outer coating layer (preferably final or taste masking coating), comprising or consisting of a mixture comprising or consisting of 50-60% (w/w) EC, 20-25% (w/w) HPMC, 17.522.5% (w/w) magnesium stearate and 0.5-3% (w/w) silicon dioxide,
   wherein the material for the outer coating layer (d) makes up a coating level of 72.5 to 77.5% (w/w).

In this embodiment the pharmaceutically active ingredient is preferably selected from cilobradine or cilobradine hydrochloride.

It is preferred that the multi-layered particles or pellets comprise appropriate flavors in the outer coating. For example it has turned out to be useful to apply meat flavors in order to make the particles attractive for carnivorous animals.

According to dissolution data, it was found that the dissolution of EC/HPMC coated particles is delayed, therefore providing efficient taste and/or odor masking of the bitter active ingredient such as cilobradine hydrochloride. This could be verified by acceptance tests with laboratory cats.

Usually, EC/HPMC films show sustained release behavior more or less independent of pH. In this case, however, the product surprisingly showed a slower release at pH 6.8 which is favorable with regard to this embodiment if taste masking is present, i.e. providing efficient taste masking in the oral cavity and a faster release in the acidic stomach. Without wishing to be bound by this theory, this may be explained by the lipophilicity profile of a specific pharmaceutically active ingredient such as cilobradine hydrochloride, which is slightly more lipophilic at neutral pH values than at acidic pH values. This, together with the coating applied, is expected to result in a slower release at neutral pH values, sufficient to use the respective multi-layered particles for the application of this drug for the treatment of animals like cats.

A preferred mode of this aspect is a multi-layered particle or pellet as described before, wherein the pharmaceutically active ingredient in its incorporated chemical form, preferably cilobradine, makes up 2 to 10% (w/w) of the final multi-layered particle (calculated as hydrochloride), preferably 3 to 7.5% (w/w), more preferred 4 to 6% (w/w), most preferred 5.25 to 5.75% (w/w).

For the preparation of multi-layered particles or pellets comprising the drug cilobradine hydrochloride it has been found to be the best solution to apply the two outermost layers (i.e. the seal coating and the final coating) by use of organic solvents. These organic solvents can, for example, be selected from acetone, methanol, ethanol or dichloromethane or mixtures thereof. The use of organic solvents may have the advantage of a faster drying step, thus shortening the time for the active ingredient to diffuse into the respective layer. Further, the choice of an organic solvent is especially preferred for pharmaceutically active ingredients with a high dissolution rate in water. The organic solvent then further hinders the diffusion of the active substance into outer layers during the layering step. Thus, by using organic solvents, it is possible to minimize migration of the active substance and thereby to ensure a very effective taste and/or odor masking. In addition, the layers that are produced with an organic solvent may be tighter than those produced with water and have an overall more even appearance. These however subtle differences may be exploited if the particles need to be optimized with respect to their dissolution behavior vs. barrier function.

A preferred method to produce the multi-layered particles or pellets takes place in a fluid bed, preferably a Wurster fluid bed coating process (Wurster process), for details it is referred to WO 2013/024023 (PCT/EP2012/065680) the whole disclosure thereof being incorporated in the present disclosure by reference.

The multi-layered particles or pellets in this embodiment may be designed to be taste-masked, especially in combination with outer coating layers (final or taste masking coating). These multi-layered particles or pellets added to the liquid oily composition, which provide the liquid dosage form, can be used per se via oral application for the treatment/prophylaxis of patients. For example they can be added to the food. The multi-layered particles or pellets provide a high mechanical stability and are physically very stable, generally so stable that they stay intact even if the patient bites on it, e.g. during the ingestion of the food stuff.

For this purpose an appropriate amount of the respective formulation provided in the oily composition as already disclosed can be added to the patient's/animal's food stuff, e.g. by the persons taking care of the animals. This is very advantageous if the pharmaceutically active ingredient such as an I_{F}-channel inhibitor is intended to be used at a low dosage for prophylaxis, in the case of an I_{F}-channel inhibitor against heart diseases, preferably heart failure.

Liquid dosage forms in general offer the option of very flexible dosing by adjusting the administered volume. Indeed, it has been found in acceptance tests that the oily suspensions (preferably flavored) containing one or multi-layered particles or pellets, lead to the desired result: they are very well accepted by the patients (exemplified by cats) and are in the majority of cases ingested voluntarily.

Furthermore, basically all sorts of additive materials that are suitable for the multi-layered particles and/or liquid dosage form can be mixed in, which are suitable to be used in the oral application route and dosage regime of the respective animal patient group.

To sum up the above discussion, the multi-layered particles according to this embodiment of the present invention are highly suitable to be used in the kit-of-parts. The multi-layered particles or pellets are optionally or preferably taste masked and at the same time they show advantageous dissolution profiles. Accordingly, the multi-layered architecture of the particles allows the formulation of palatable pharmaceutical compositions for preferable use in veterinary medicine, even with the use of pharmaceutically active ingredients such as I_{F}-channel inhibitors that could otherwise be rejected by taste-sensitive patients. Thus the acceptance and compliance of the dosage form provided is ameliorated. The features, especially the mechanical stability of the multi-layered particles further allow the possibility for accurate dose adjustment in an overall liquid form.

Therefore, the kit-of-parts according to the present invention provides a liquid dosage form ready to be administered to a patient/an animal patient. The finally prepared and obtained liquid dosage form is administered once, twice or three times per day, preferably once or twice, even more preferred once per day.

It is generally known that the dosage regimen for a pharmaceutically active ingredient will, of course, vary depending upon known factors, such as the pharmacodynamic characteristics of the particular agent and its mode and route of administration; the species, age, sex, health, medical condition, and weight of the patient; the nature and extent of the symptoms; the kind of concurrent treatment; the frequency of treatment; the renal and hepatic function of the patient, and the effect desired.

Thus, a physician or veterinarian can determine and prescribe the effective amount of the drug required to prevent, counter, or arrest the progress of the disorder. The term “effective amount” as used herein means an amount sufficient to achieve an alleviation of a disease in a patient when a pharmaceutically active ingredient is given. The progress of the therapy can be monitored by standard diagnosis. The progress of the therapy can also be monitored by clinical symptoms as well as quality of life related parameters as defined above herein. Furthermore the effective amount also gives the patient, preferably feline patients, even more preferred cats, an improved quality of life and/or improved general health condition and/or prolonged life expectancy. This progress and improvement of well-being obtained by the therapy can be monitored by the pet owner as well as the veterinarian.

By way of general guidance, the dosage per administration of the active ingredient, i.e. the I_{F}-channel inhibitor or a pharmaceutically acceptable salt thereof, in particular cilobradine, zatebradine, alinidine or ivabradine, preferably cilobradine or zatebradine, even more preferred cilobradine and cilobradine hydrochloride, when used for the treatment and/or prevention of a heart disease, particularly heart failure, will range from 0.01 mg/kg bodyweight to 1.0 mg/kg bodyweight per administration, preferably 0.02 to 0.8 mg/kg bodyweight, more preferred from 0.04 to 0.6 mg/kg bodyweight, even more preferred from 0.06 mg/kg to 0.4 mg/kg bodyweight, 0.08 mg/kg to 0.3 mg/kg bodyweight, most preferred from 0.1 mg/kg bodyweight to 0.3 mg/kg bodyweight or 0.1 to 0.2 mg/kg bodyweight per administration. These dosages should be administered once or twice per day. The treatment is advisable in clinically apparent cases, both in acute as well as in chronic settings.

Therefore, the amount of the solid coated one or multi-layered particles or pellets as well as the amount of the oily composition is selected accordingly in order to provide a suitable dose in the produced liquid dosage form provided in the kit-of-parts according to the present invention.

In a further aspect of the present invention the kit-of-parts be provided as a set of several kit-of-parts, wherein a defined number of each of the components A) to E) is provided, for example two kit-of-parts may be provided, each kit-of-parts comprising components A) to E), respectively, as defined above.

Further, a combined therapy of a pharmaceutically active ingredient such as an I_{F}-channel inhibitor and a second active therapeutic agent is known. Therefore, the kit-of-parts according to the present invention may be combined with a packaging unit comprising a second active therapeutic agent, which in the case the pharmaceutically active ingredient is an I_{F}-channel inhibitor is selected from the group consisting of diuretics, calcium channel blockers, beta-adrenoreceptor antagonists, positive inotropes, ACE inhibitors, anti-thrombotic agents and antiarrhythmic agents, for the treatment and/or prevention of a patient, preferably feline patient, even more preferred a cat, suffering from heart disease, preferably heart failure.

While this invention is susceptible of embodiment in many different forms, this specification and the accompanying drawings disclose only some specific forms as examples of the invention. The invention is not intended to be limited to the embodiments so described, however. The scope of the invention is pointed out in the appended claims. Figures illustrating the components show some mechanical elements that are known and that will be recognized by one skilled in the art. The detailed descriptions of such known elements are not necessary to an understanding of the invention, and accordingly, are herein presented only to the degree necessary to facilitate an understanding of the novel features of the present invention.

Therefore, the accompanying drawings are included to provide a further understanding of the disclosure, and are incorporated into and constitute a part of this specification. The drawings illustrate various embodiments of the disclosure, and together with the description serve to explain the principles and operations of the disclosure.

The various elements depicted in the drawings are merely representational and are not necessarily drawn to scale. Certain sections thereof may be exaggerated, while others may be minimized. The drawings are intended to illustrate exemplary embodiments of the disclosure that can be understood and appropriately carried out by those of ordinary skill in the art.

In the accompanying drawings that form part of the specification, the same reference numbers are employed to designate the same elements throughout the same.

### BRIEF DESCRIPTION OF THE DRAWINGS

Further advantages, features, characteristics and aspects of the present invention arise from the following description of preferred embodiments along with the drawings which show as follows:

FIG. 1 a schematic view of an exemplary embodiment of a package as component A) of the kit-of-parts according to the present invention;

FIG. 2 a schematic view of an exemplary embodiment of a container as component B) of the kit-of-parts according to the present invention;

FIG. 3 a schematic perspective view of an exemplary embodiment of a gasket seal present in a sealing of a container as component B) of the kit-of-parts according to the present invention;

FIG. 4 a schematic side view of an exemplary embodiment of a dispenser as component C) of the kit-of-parts according to the present invention;

FIG. 5A a sectional view of an exemplary embodiment of a sealing (cap) of a dispenser as component C) of the kit-of-parts according to the present invention;

FIG. 5B a sectional view of a further exemplary embodiment of a sealing (cap) of a dispenser as component C) of the kit-of-parts according to the present invention;

FIG. 6A a schematic plan view of an exemplary embodiment of an adapter as component D) of the kit-of-parts according to the present invention;

FIG. 6B a schematic perspective view of an exemplary embodiment of an adapter as component D) of the kit-of-parts according to the present invention;

FIG. 6C a sectional view of a further exemplary embodiment of an adapter as component D) of the kit-of-parts according to the present invention;

FIG. 7 a schematic side view of an exemplary embodiment of a container B) to which an adapter as component D) of the kit-of-parts according to the present invention is applied;

FIG. 8 a schematic side view of an exemplary embodiment of a syringe-like dispenser as component C) (in part) inserted in the adapter as component D) applied in the container B) of the kit-of-parts according to the present invention.

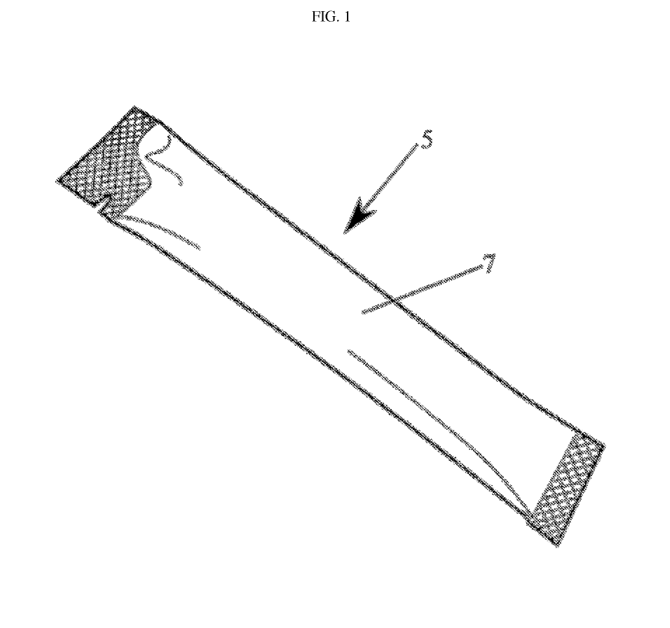

FIG. 1 shows a schematic view of an exemplary embodiment of a package 5 as component A) of the kit-of-parts according to the present invention. Package 5 may be selected from a wide range of packages to be used in the pharmaceutical field. The package 5 has an arbitrary size and form and provides a cavity 7 wherein the solid coated one or multi-layered particles or pellets are taken up and accommodated. The exemplary embodiment shown in FIG. 1 is a package 5 comprising a laminated film composed of 2 foils having a tube-like form wherein the solid formulation is contained. The respective form, size and built-up of package 5 shown in FIG. 1 are only for illustration purposes and are not limiting, other forms, dimensions and designs are possible.

The mechanism how to open the package 5 and to discharge the solid formulation contained is not limited and depends from the selected type and material of the package 5. For example, the package may be opened by hand or may be cut off by scissors or a knife.

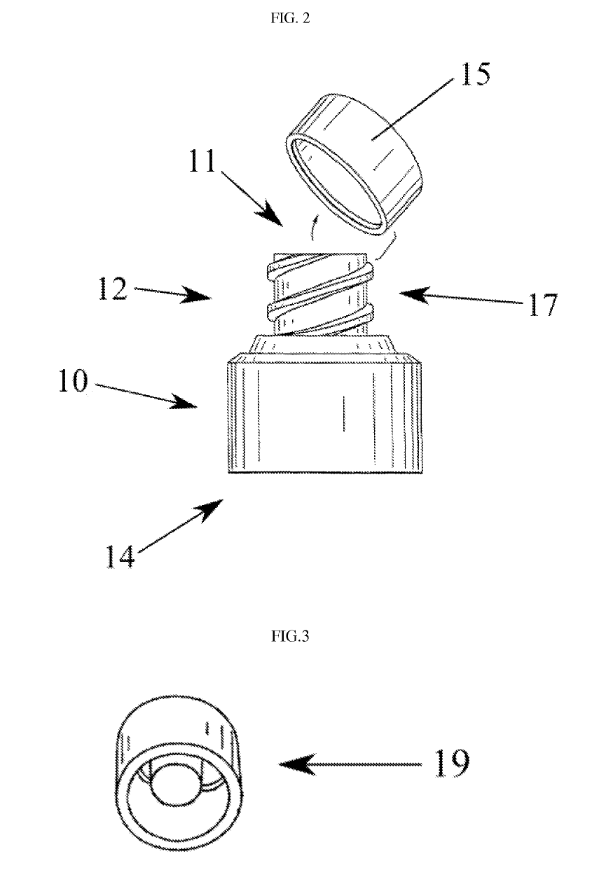

FIG. 2 shows a schematic view of an exemplary embodiment of a container 10 as component B) of the kit-of-parts according to the present invention. The shown container 10 has a cylindrical form having a hollow body, the cross-section of the container 10 is, for example, formed circular or oval. Other cross-sections are possible.

Container 10 serves to the uptake and storage of the liquid oily composition (not shown) which is extractable from the opening 11 disposed at the upper end portion 12 of container 10. The opening 11 is disposed in the mouthpiece of container 10. The bottom end portion 14 is formed as a flat basement so that the container 10 may be readily deposited. Other configurations may be possible.

The container 10 may be preferably a glass vial or a glass bottle.

At the upper end 12 of the container 10 the sealing 15 thereof has just been removed in FIG. 2. The sealing 15 may have a suitable form to seal the container 10. In the present exemplary embodiment the sealing 15 may be a screw cap. The container 10 may have respective means 17 in order to apply and secure the sealing 15 in a suitable manner. For example, the container 10 may include a threaded female connection at the upper end 12 that cooperates with a male connection of a screw-top cap (not shown) to securely connect both. Other types of sealing mechanism are also possible. In addition, a child-proof lock may be used.

According to a preferred embodiment (not shown) of the present invention the sealing 15 provided on the container 10, in the present embodiment a screw cap, is designed and dimensioned in such a manner that the container 10 may be sealed again with the sealing 15 if an adapter 50 is already mounted on the container 10, whereby the sealing 15 is in a usual manner so as to prevent the outflow of the content.

The respective form, size and seal of container 10 shown in FIG. 2 are only for illustration purposes and are not limiting, other forms, dimensions and designs are possible.

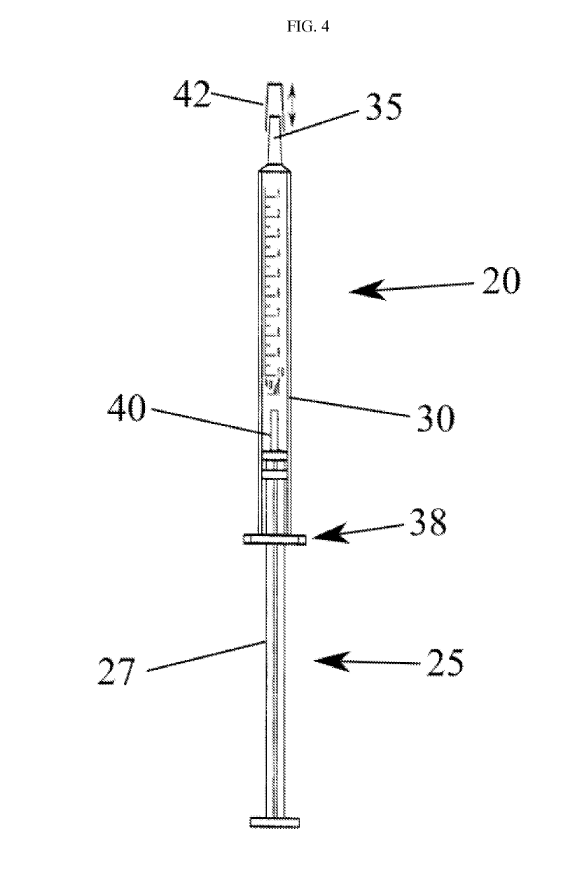

FIG. 3 is a schematic perspective view of an exemplary embodiment of a gasket seal 19 usually present in the inside space of a sealing 15 such as a screw cap of a container 10 as component B) of the kit-of-parts according to the present invention. The gasket seal 19 may have any suitable size and form in order to provide sufficient impermeability to the container 10. The preferred material of the gasket seal 19 is a plastic material. The dimension and form of such a gasket seal 19 is preferably adapted in such a manner that a sealing 15 provides a fluid-tight connection with the container 10 in the closed state even if an adapter 50 is mounted on the container 10.

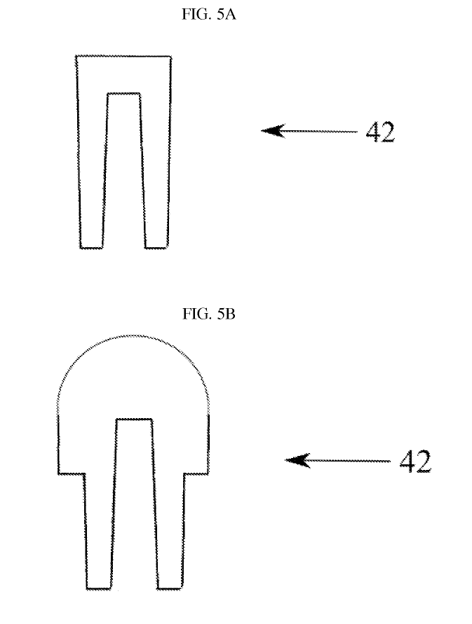

FIG. 4 illustrates a schematic side view of an exemplary embodiment of a dispenser 20, preferably syringe-like dispenser as component C) of the kit-of-parts according to the present invention.

The syringe-like dispenser 20 includes a syringe barrel 30 having an elongated cylindrical body defining a chamber for retaining fluid, a plunger 25 in fluid-tight slidable engagement inside the barrel 30, an elongated plunger rod 27 extending in the longitudinal direction through the barrel 30, and a tip 35 having a tip passageway therethrough in fluid communication with the chamber. Furthermore, the dispenser 20 comprises a usually known finger support or flange 38.

The front surface of the dispenser 20 is preferably provided with a scale reading which facilitates to determine and adjust the dosage of the liquid dosage form containing a defined amount of pharmaceutically active ingredient to be administered to the patient/animal patient. In the exemplary embodiment shown the labeling indicated on the dispenser 20 contains a number of marks which preferably correspond to the weight (in kg) of the patient/animal patient. For example, the division scale may be classified in 5 different marks from 1 to 5 kg, each mark represents the amount in kg, respectively increasing 1 kg from mark to mark. Therefore, in case the user has a cat having 4 kg weight, the dispenser shall be filled with the pharmaceutical liquid dosage form up to the mark ‘4 kg’ in order to provide the correct dose to be administered.

In the exemplary embodiment the plunger 25 of the dispenser 20 has a protruding part 40 which extends in the direction of the tip 35 and fits within the tip passageway in such a manner to be able to press the liquid in the tip passageway practically completely out of the dispenser 20. This embodiment ensures that the liquid dosage form contained in the dispenser 20 is in total administered to the animal to be treated and no product is remaining in the dispenser 20 after administration which could be contaminated later on.

In the shown embodiment a cap 42 may be used to seal the dispenser 20 after use in order to avoid contamination of the interior of the dispenser 20, particularly in case of a repeated use thereof. A sealing of the dispenser is also advantageous in order to prevent the ingress of humidity, particularly in case the pharmaceutically active ingredient present is sensitive against moisture.

The sealing of the dispenser 20 is not limited according to the present invention, any type of sealing known by the skilled person may be used.

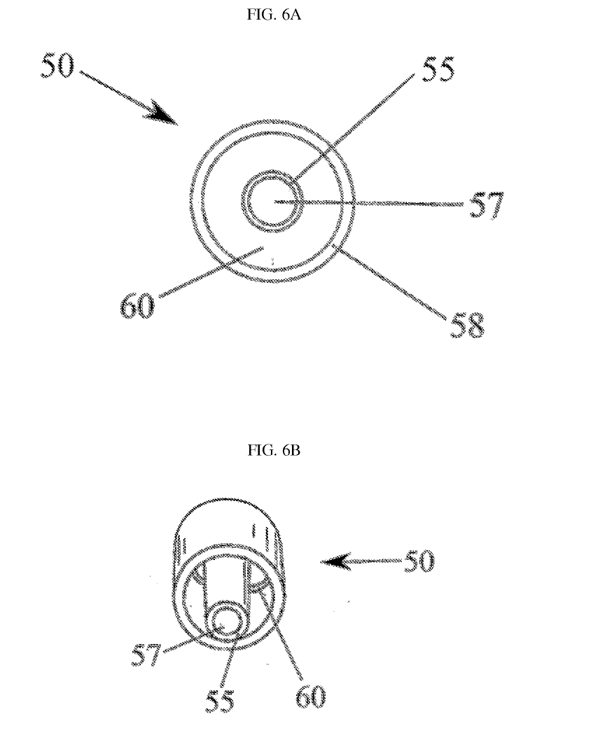

FIGS. 5A and 5B, respectively, illustrate a sectional view of an exemplary embodiment of a sealing such as a cap 42 of a dispenser 20 as component C) of the kit-of-parts according to the present invention.

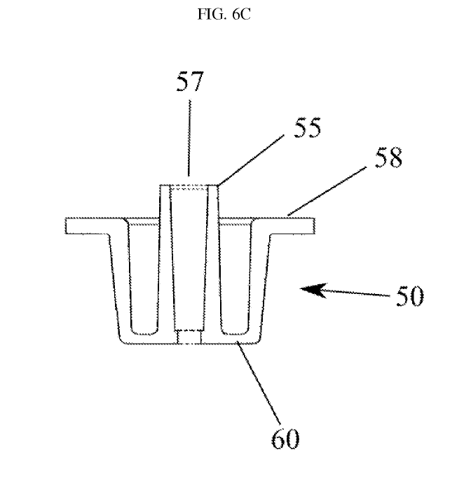

FIG. 6A shows a schematic plan view, and FIG. 6B shows a schematic perspective view of an exemplary embodiment of an adapter 50 as component D) of the kit-of-parts according to the present invention. FIG. 6C shows a further exemplary embodiment of an adapter 50 as component D) of the kit-of-parts according to the present invention in a sectional view.

In FIGS. 6A to 6C the adapter 50, respectively, has an application arrangement piece 55 with a through-hole 57 to fit with a dispenser 20 to the container 10. On one hand, the form of the adapter 50 is adapted to the form of the opening 11 and mouthpiece of the container 10 so that the adapter 50 is a removable seal disposed on, and sealingly engaged with a part of the inner walls at the upper end 12 of container 10. On the other hand, the adapter 50, particularly the through-hole 57 of the application arrangement piece 55, is adapted to the form of the tip 35 of a dispenser 20 to be inserted. Therefore, the adapter 50 is formed to couple the container 10 with the dispenser 20. In the exemplary embodiments shown the adapter 50 is a stopper, preferably made of a plastic material.

The application arrangement piece 55 with a through-hole 57 in the adapter 50 provides access to the content of the container, which may extend from an end surface 60 of the adapter 50 and, in mounting position, extends in the opposite direction to the basement (bottom or lower end 14) of the container 10. The application arrangement piece 55 with a through-hole 57 may protrude from the remaining part of the adapter 50 as shown in FIG. 4a but this is not necessary in any case.

Furthermore, the adapter 50 preferably has an additional edge 58, preferably a protruding edge 58 as a further contact surface to provide additional contact with the container 10, to provide a tighter fit and to improve the fitting accuracy with the container 10.

The outer contour of the adapter 50 may be formed and adapted (not shown) in order to provide a form-fitting connection.

In the exemplary embodiments shown in FIGS. 6A, 6B and 6C the adapter 50 is provided as a rotationally symmetrical element having a circular cross-section. Other configurations may be used.

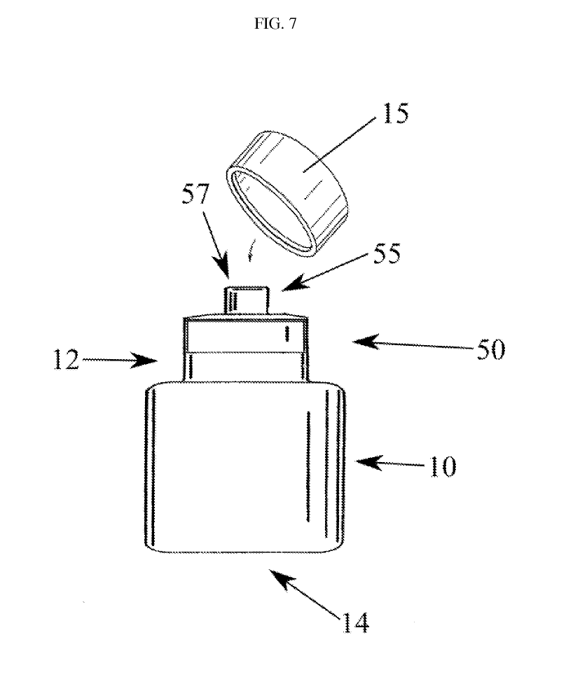

FIG. 7 shows a schematic side view of an exemplary embodiment of a container 10 as component B) to which an adapter 50 as component D) of the kit-of-parts according to the present invention is applied. The adapter 50 is applied on the container 10 so that the application arrangement piece 55 with the through-hole 57 provides easy access to the content of the container 10 and the sealing 15 may be used to close the container 10 again whereby the adapter 50 still remains on the container. The sealing 15 of the container 10 may comprise a gasket seal 19 provided in the inner surface (not shown) in order to provide accurate fit and a fluid-tight connection even if the adapter 50 is present.

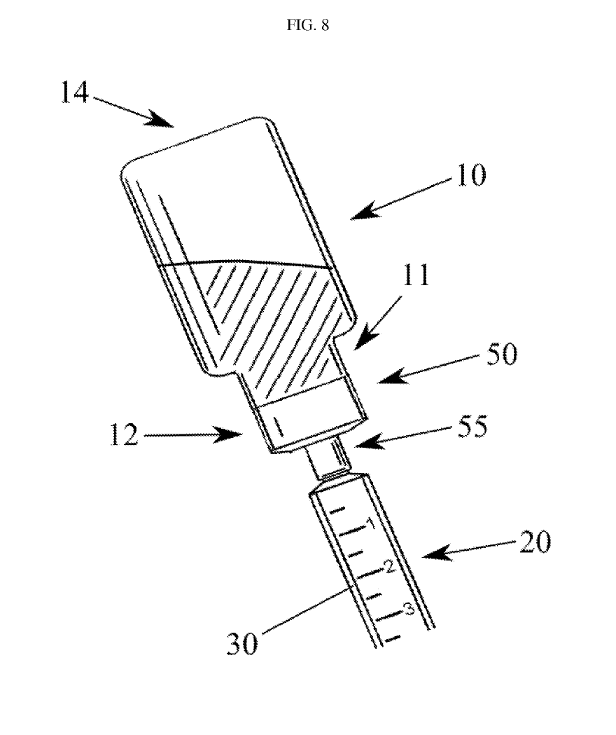

FIG. 8 shows a schematic side view of an exemplary embodiment of a dispenser 20 (in part) as component C) inserted in the adapter 50 as component D) applied in the container 10 as component B) of the kit-of-parts according to the present invention.

In the exemplary embodiment shown the dispenser 20 is inserted into the through-hole 57 of the application arrangement piece 55 in order to remove the liquid dosage form out of the container 10. The application arrangement piece 55 and the trough-hole 57 are formed to accommodate and surround the tip 35 of the dispenser 20. The form and size of both is matched to each other in such a way that the withdrawing of the liquid dosage form is possible without leakage of the content of the container 10, whereby the container 10 is held upside down with the opening 11 downwards.

In the following the invention shall be illustrated in form of exemplary formulations. However, the present invention is not limited to the described formulations, but other dosage forms and additives are possible.

### EXAMPLES

### a) Solid Coated Multi-Layered Particles (of Package A)

In the following an example of multi-layered particles provided in the kit-of-parts in the package A) is given in detail. The pharmaceutically active ingredient used is a representative compound of the chemical class of cyclic amine derivatives, i.e. of bitter tasting cilobradine in the respective hydrochloride form. For details of the syntheses of these multi-layered particles it is referred to WO 2013/024023 (PCT/EP2012/065680) the whole disclosure thereof being incorporated in the present disclosure by reference.

TABLE 1
Detailed composition of multi-layered particles comprising (+)-3-
[N-(2-(3,4-dimethoxy-phenyl)ethyl)-piperidin-3-(S)-yl)-methyl]-
(7,8-dimethoxy-1,3,4,5-tetrahydro-2H-3-benzazepin-2-one hydrochloride

| | Amount | |
|---|---|---|
| Component | [% (w/w)] | Function |
| Inert core particles | 35.23 | Carrier |
| (CELLETS ® 100) | | |
| Pharmaceutically active | 5.53 | Drug layer |
| ingredient cilobradine | | |
| (calculated as | | |
| hydrochloride) | | |
| HPMC | 2.63 | |
| Polyvinylpyrrolidone K 30 | 10.05 | Seal coating |
| Talc | 3.01 | |
| Ethylcellulose | 23.86 | Final coating (taste |
| HPMC | 10.25 | masking) |
| Magnesium stearate | 8.68 | |
| Colloidal silicium dioxide | 0.78 | Reduction of electrostatic |
| (AEROSIL ® 200) | | charging in seal coating |
| | | and final coating |

### b) Liquid Pharmaceutical Composition (of Container B)

In the following an example of liquid oily composition which is present in container B) of the kit-of-parts according to the present invention is given in detail. The liquid oily composition in the present example consisted of a mixture of medium chain triglycerides (MIGLYOL® 821, bought from Sasol, Hamburg, Germany), a hydrophilic colloidal silicon dioxide (AEROSIL® 200, Evonik), a hydrophobic colloidal silicon dioxide (AEROSIL® R972, Evonik) and meat flavor, at the weight ratios listed in the following table 2.

TABLE 2
Liquid pharmaceutical composition

| Component | Amount [% (w/w)] |
|---|---|
| Medium chain triglycerides (MIGLYOL ® | 93.23 |
| 821) | |
| Hydrophilic colloidal silicium dioxide | 4.44 |
| (AEROSIL ® 200) | |
| Hydrophobic colloidal silicium dioxide | 1.82 |
| (AEROSIL ® R972) | |
| Meat flavor | 0.51 |

### c) Ready to Use Liquid Dosage Form

### Solid Coated Multi-Layered Particles+Liquid Oily Composition

In the following an example of a ready to use liquid dosage form which may be produced with the kit-of-parts of the present invention is given.

In order to provide the liquid dosage form the exemplary solid coated multi-layered particles as indicated above under a), which are taste masked, were suspended in the liquid composition indicated above under b) to an amount of about 3.8% (w/v), resulting in a concentration of 2 mg/ml of the pharmaceutically active ingredient (calculated as hydrochloride).

It has been found that the composition of the oily composition, especially the mixture of hydrophilic and hydrophobic colloidal silica, ensures appropriate viscosity behavior of the suspension that remains more or less unchanged over the storage period. During storage, the suspension exhibits a high viscosity, preventing sedimentation of the suspended cilobradine particles. If shaken, the viscosity of the suspension is transiently lowered so that it can easily be applied via an oral dispenser, preferably a syringe-like oral dispenser.

The ready to use liquid dosage form provided with the kit-of-parts of the present example may be directly used for the treatment and/or prevention of heart disease in patients, preferably animals.

It will be apparent to those skilled in the art that various modifications and variations can be made to the present disclosure without departing from the spirit and scope of the disclosure. Thus it is intended that the present disclosure cover the modifications and variations of this disclosure provided they come within the scope of the appended claims and their equivalents.

### LEGEND

5 package
7 cavity
10 container
11 opening
12 upper end
14 bottom end
15 sealing
17 screw thread
19 gasket seal in the sealing of the container
20 dispenser
25 plunger
27 plunger rod
30 barrel
35 tip
38 finger support or flange
40 protruding part
42 cap of the dispenser
50 adapter
55 application arrangement piece
57 through-hole
58 edge
60 end surface

1. A kit-of-parts comprising:
a) a packaging, containing a pharmaceutically active ingredient in the form of solid coated one or multi-layered particles;
b) a container, comprising a liquid oily composition;
c) a dispenser;
d) an adapter, having an application arrangement piece with a through-hole to fit the dispenser to the container of B); and
e) instructions for use of the kit.

2. The kit-of-parts according to claim 1, wherein the solid coated multi-layered particles of A) comprise:
a) an inert core,
b) one or more coating layer(s), comprising the pharmaceutically active ingredient and a binder,
c) an intermediate coating layer free from a low molecular weight water-soluble ionic compound, preferably comprising a water-soluble pharmaceutical film-forming compound in form of poly(1-vinylpyrrolidin-2-one) (PVP), and
d) an outer coating layer free from a low molecular weight water-soluble ionic compound, preferably comprising a mixture comprising 60-90% (w/w) ethylcellulose (EC) and 10-40% (w/w) hydroxypropyl methylcellulose (HPMC).

3. The kit-of-parts according to claim 1, wherein the container is a bottle, flask, vial, ampoule, pharmaceutical phial, pouch, preferred a vial or a bottle, particularly preferred a glass vial or bottle.

4. The kit-of-parts according to claim 1, wherein the liquid oily composition in the container comprises
a) an edible oil, such as a vegetable oil, preferably selected from linseed oil, peanut oil, sunflower oil, olive oil, safflower oil or the like; or
b) an animal oil such as fish oil, or
c) one triglyceride or a mixture of several triglycerides, or
d) mixtures thereof,
preferably the container comprises a mixture of triglycerides, more preferably of medium chain triglycerides.

5. The kit-of-parts according to claim 1, wherein the liquid oily composition in the container comprises one or more viscosity enhancers selected from the following: silicon dioxide(s), preferably colloidal silicon dioxide(s), selected from the group consisting of: hydrophilic colloidal silicon dioxide, hydrophobic silicon dioxide, EC (cellulose ether), poly(1-vinylpyrrolidin-2-one) (PVP), aluminum stearate, xanthan gum, carrageen, and starch derivatives; preferably the liquid oily composition in the container comprises a colloidal silicon dioxide, selected from the group consisting of: hydrophilic colloidal silicon dioxide, hydrophobic silicon dioxide, and mixtures thereof; more preferably a mixture of colloidal silicon dioxide, consisting of hydrophilic colloidal silicon dioxide and hydrophobic silicon dioxide.

6. The kit-of-parts according to claim 1, wherein the liquid oily composition in the container comprises a mixture of colloidal silicon dioxide, comprising hydrophilic colloidal silicon dioxide and hydrophobic colloidal silicon dioxide, preferably at a weight percent ratio of 0.5:1 to 50:1, more preferred is 1:1 to 25:1, particularly preferred is 2:1 to 10:1, most preferred is 2.25:1 to 5:1.

7. The kit-of-parts according to claim 1, wherein the liquid oily composition comprises a mixture of medium chain triglycerides, a colloidal silicon dioxide, optionally a hydrophobic colloidal silicon dioxide and preferably one or more flavoring agents such as meat flavor.

8. The kit-of-parts according to claim 1, wherein the dispenser comprises a labeling on the front surface of the dispenser comprising marks which correspond to the weight in kilograms of the animal patient to be treated so that the pharmaceutical liquid dosage form may be filled into the dispenser according to the animal's weight and provides the correct dose to be administered to the animal.

9. The kit-of-parts according to claim 1, wherein the solid coated multi-layered particles comprise:
a) an inert core, comprising microcrystalline cellulose,
b) one coating layer, comprising 60 to 70% (w/w) of the pharmaceutically active ingredient, 25 to 35% (w/w) of HPMC as binder and 0.53% (w/w) magnesium stearate,
c) an intermediate coating layer, comprising 70-80% (w/w) PVP K 30, 20-25% (w/w) talc and 0.5-5% (w/w) silicon dioxide and
d) an outer coating layer, comprising a mixture, comprising 50-60% (w/w) EC, 20-25% (w/w) HPMC, 17.522.5% (w/w) magnesium stearate and 0.5-3% (w/w) silicon dioxide,
wherein the material for the outer coating layer (d) makes up a coating level of 72.5 to 77.5% (w/w).

10. The kit-of-parts according to claim 1, wherein the instructions of component E) comprise the following:
i) adding the whole content of packaging into the container;
ii) shaking the container; and
iii) removing liquid from the container.

11. The kit-of-parts according to claim 1, wherein the instructions of component E) comprise the following:
a) opening of the container;
b) opening of the packaging;
c) adding the whole content of the packaging into the container;
d) applying the adapter, having an application arrangement piece with a through-hole onto the container;
e) closing the container;
f) shaking the container;
g) opening the container;
h) inserting the tip of a dispenser into the through-hole of the application arrangement piece of the adapter;
i) removing liquid from the container, using the dispenser;
j) disconnecting the dispenser from the adapter;
k) administering the liquid with/by means of the dispenser to the patient/animal or to patient/animal food;
l) closing the container;
m) optionally repeating instructions f) to l);
whereby the sequence of instruction a) and instruction b) may also be reversed,
whereby the sequence of instruction k) and instruction 1) may also be reversed.

12. The kit-of-parts according to claim 1, wherein the solid coated one or multi-layered particles are taste-masked.

13. The kit-of-parts according to claim 1, wherein the pharmaceutically active ingredient of the solid coated one or multi-layered particles is selected from the class of I_{F}-channel blockers.

14. The kit-of-parts according to claim 1, wherein the I_{F}-channel blocker is zatebradine (1-(7,8-dimethoxy-1,3,4,5-tetrahydro-2H-3-benzazepin-2-on-3-yl)-3-[N-methyl-N-[(2-(3,4-dimethoxyphenyl)ethyl]amino]-propane), or 3-[(N-(2-(3,4-dimethoxy-phenyl)-ethyl)piperidin-3-yl)-methyl]-(7,8-dimethoxy-1,3,4,5-tetrahydro-2H-3-benzazepin-2-on, or cilobradine ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on) or alinidine (2-(N-allyl-2,6-dichloro-anilino)-2-imidazoline), preferably cilobradine, mostly preferred cilobradine hydrochloride ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-one hydrochloride).

15. The kit-of-parts according to claim 1, wherein the solid coated one or multi-layered particles of A) comprise 0.001 to 0.2 mg of the I_{F}-channel inhibitor or the pharmaceutically acceptable salt thereof per mg of the solid coated one or multi-layered particles.

16. The kit-of-parts according to claim 13, wherein the kit-of-parts is combined with a packaging unit comprising a second active therapeutic agent selected from the group consisting of diuretics, calcium channel blockers, beta-adrenoreceptor antagonists, positive inotropes, ACE inhibitors, anti-thrombotic agents and antiarrhythmic agents.

17. The kit-of-parts according claim to 14, wherein the kit-of-parts is combined with a packaging unit comprising a second active therapeutic agent selected from the group consisting of diuretics, calcium channel blockers, beta-adrenoreceptor antagonists, positive inotropes, ACE inhibitors, anti-thrombotic agents and antiarrhythmic agents.

18. The kit-of-parts according to claim 15, wherein the kit-of-parts is combined with a packaging unit comprising a second active therapeutic agent selected from the group consisting of diuretics, calcium channel blockers, beta-adrenoreceptor antagonists, positive inotropes, ACE inhibitors, anti-thrombotic agents and antiarrhythmic agents.

19. The kit-of-parts according to claim 1, wherein the dispenser is syringe-like.

20. The kit-of-parts according to claim 19, wherein the syringe-like dispenser comprises a syringe barrel having an elongated cylindrical body, defining a chamber for retaining fluid, a plunger in fluid-tight slidable engagement inside the barrel, an elongated plunger rod, extending in the longitudinal direction through the barrel, and a tip, having a tip passageway therethrough in fluid communication with the chamber, preferably the syringe-like dispenser is a dispensing pipette.

21. The kit-of-parts according to claim 1, for the use in a method of treating and/or preventing a heart disease in a mammalian patient, preferably in a companion animal, more preferred in a feline, in particular in a cat.

22. A method for administering a pharmaceutically active ingredient in the form of solid coated one or multi-layered particles, comprising the steps of:
a) opening of a container comprising a liquid oily composition;
b) opening of a packaging comprising the active ingredient in the form of solid coated one or multi-layered particles;
c) adding the whole content of the packaging into the container;
d) applying an adapter having an application arrangement piece with a through-hole onto the container;
e) closing the container;
f) shaking the container;
g) opening the container;
h) inserting the tip of a dispenser into the through-hole of the application arrangement piece of the adapter;
i) removing liquid from the container, using the dispenser;
j) disconnecting the dispenser from the adapter;
k) administering the liquid with/by means of the dispenser to the patient/animal or to patient/animal food;
l) closing the container;
m) optionally repeating steps f) to l);
whereby the sequence of step a) and step b) may also be reversed,
whereby the sequence of step k) and step l) may also be reversed.

23. The method according to claim 22, wherein the solid coated one or multi-layered particles are taste-masked.

24. The method according to claim 22, wherein the pharmaceutically active ingredient of the solid coated one or multi-layered particles is selected from the class of I_{F}-channel blockers, preferably cilobradine ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on) or a pharmaceutically acceptable salt thereof.

25. The method according to claim 23, wherein the pharmaceutically active ingredient of the solid coated one or multi-layered particles is selected from the class of I_{F}-channel blockers, preferably cilobradine ((+)-3-[(N-(2-(3,4-dimethoxy-phenyl)ethyl)-piperidin-3-(S)-yl)-methyl]-(7,8-dimethoxy-1,3,4,5-tetrahydro-2H-3-benzazepin-2-on) or a pharmaceutically acceptable salt thereof.

26. The method according to claim 22, wherein the dispenser is syringe-like.
